(19) United States
(12) Patent Application Publication
(10) Pub. No.: US 2011/0144156 A1
(43) Pub. Date: Jun. 16, 2011

(54) A3 ADENOSINE RECEPTOR ALLOSTERIC MODULATORS

(75) Inventors: Adriaan P. Ijzerman, Haarlem (NL); Aniko Goblyos, Ujszasz (HU); Johannes Brussee, Rijnsburg (NL)

(73) Assignees: UNIVERSITEIT LEIDEN, Leiden (NL); CAN-FITE BIOPHARMA LTD., Petach Tikva (IL)

(21) Appl. No.: 13/059,836

(22) PCT Filed: Aug. 12, 2009
(86) PCT No.: PCT/IL2009/000791

§ 371 Date: Feb. 18, 2011

Related U.S. Application Data
(60) Provisional application No. 61/136,214, filed on Aug. 19, 2008.

(51) Int. Cl. A61K 31/4709; A61K 31/47; C07D 215/38; A61P 35/00

(52) U.S. Cl. 514/314; 514/313; 546/159

(57) ABSTRACT

Provided are 2,4-disubstituted quinoline derivatives that are A3 adenosine receptor modulators (A₃RM), for use in the treatment of a condition which is treatable by adenosine, an A₃ adenosine receptor (A₃AR) agonist, or an A₃ adenosine receptor antagonist. The 2,4-disubstituted quinoline derivatives can be used in the treatment of a condition treatable by an adenosine or an A₃AR agonist, by enhancing activity of a protein (by binding of said 2,4-disubstituted quinoline derivative to the A₃AR). Some conditions treatable by the 2,4-disubstituted quinoline derivative when used for enhancing activity include, malignancy, an immuno-compromised affliction, high intraocular pressure, or a condition associated with high intraocular pressure. Further provided is a method for treatment of a subject having a condition treatable by adenosine, an A₃AR agonist, or an A₃AR antagonist making use of the defined 2,4-disubstituted quinoline derivative (s), and pharmaceutical composition and comprising said derivative and a kit comprising the derivative and instructions for use thereof. One specific 2,4-disubstituted quinoline derivative includes N-{2-[(3,4-dichlorophenyl)amino]quinolin-4-yl}cyclohexanecarboxamide. The 2,4-disubstituted quinoline derivatives can be formulated in a form suitable for oral administration.

### FIELD OF THE INVENTION

This invention relates to A₃ adenosine receptor (A₃AR) allosteric modulators and uses thereof.

### PRIOR ART

The following is a list of prior art which is considered to be pertinent for describing the state of the art in the field of the invention. Acknowledgement of these references herein will at times be made by indicating their number within brackets from the list below.
1. Fishman P, et al. Evidence for involvement of Wnt signaling pathway in IB-MECA mediated suppression of melanoma cells. Oncogene., 21:4060-4064 (2002).
2. Fishman P, et al. Targeting the A₃ adenosine receptor for cancer therapy: inhibition of Prostate carcinoma cell growth by A₃AR agonist. Anticancer Res., 23:2077-2083 (2003).
3. Madi L, et al. A3 adenosine receptor activation in melanoma cells: association between receptor fate and tumor growth inhibition. J. Bio. Chem., 278:42121-42130 (2003).
4. Ohana G, et al. Inhibition of primary colon carcinoma growth and liver metastasis by the A3 adenosine receptor agonist IB-MECA. British J. Cancer., 89:1552-1558 (2003).
5. Fishman P, et al. An agonist to the A₃ adenosine receptor inhibits colon carcinoma growth in mice via modulation of GSK-313 and NF-κB. Oncogene, 23:2465-2471 (2004).
6. US Patent Application No. 2004016709 A1.
7. Szabo, C., et al. Suppression of macrophage inflammatory protein (MIP)-1α production and collagen-induced arthritis by adenosine receptor agonists. British J. Pharmacology, 125:379-387 (1998).
8. Mabley, J., et al. The adenosine A₃ receptor agonist, N⁶-(3-iodobenzyl)-adenosine—5′-N-methyluronamide, is protective in two murine models of colitis. Europ. J. Pharmacology, 466:323-329 (2003).
9. Baharav, E., et al. The effect of adenosine and the A₃ adenosine receptor agonist IB-MECA on joint inflammation and autoimmune diseases models. Inter. J. Mol. Med. 10 (supplement 1) page 5104, abstract 499 (2002).
10. PCT Application, publication No. WO2005/0063246, entitled “Method for Treatment of Multiple Sclerosis”.
11. Montesinos, M. Carmen, et al. Adenosine A_{2A} or A₃ receptors are required for inhibition of inflammation by methotrexate and its analog MX-68. Arthritis & Rheumatism, 48:240-247 (2003).
12. Madi L, et al. The A₃ Adenosine Receptor is Highly Expressed in Tumor vs. Normal Cells: Potential Target for Tumor Growth Inhibition. Clinical Cancer Research, 10: 4472-4479 (2004).
13. US Patent Application, publication No. 20040137477 A1, entitled “A3AR as a marker for a diseased state”.
14. Gessi, S. et al. Elevated expression of A₃ adenosine receptors in human colorectal cancer is reflected in peripheral blood cells Clinical Cancer Research 10:5895-5901 (2004).
15. Birdsall N J et al., Allosteric regulation of G-protein-linked receptors Biochem Soc Trans 23:108-111 (1995).
16. Holzgrabe U and Mohr K, Allosteric modulators of ligand binding to muscarinic acetylcholine receptors, Drug Disc Today 3:214-222 (1998).
17. Bruns R F and Fergus J H, Allosteric enhancement of adenosine A₁ receptor binding and function by 2-amino-3-benzoylthiophenes, Mol Pharmacol 38:939-949 (1990).
18. Bhattacharya S and Linden J, Effects of long-term treatment with the allosteric enhancer, PD81,723, on Chinese hamster ovary cells expressing recombinant human A₁ adenosine receptors, Mol. Pharmacol 50:104-11 (1996).
19. Gao Z G, Kin S G, Soltysiak K A, Mehnan N, IJzerman A P, Jacobson K A, Selective allosteric enhancement of agonist binding and function at human A₃ Adenosine receptors by a series of imidazoquinoline derivatives, Mol Pharmacol 62:81-89 (2002).

### BACKGROUND OF THE INVENTION

G protein-coupled receptors (GPCRs) class is the largest family of cell-surface receptors which plays a crucial role in intracellular signal transduction. Adenosine receptors are part of the GPCR class, which belongs to the Class A or rhodopsin-like subfamily of GPCRs. Adenosine, a purine nucleoside, produces numerous physiological actions via cell surface adenosine receptors. These receptors are widely distributed throughout the body and are divided into four subclasses, A₁, A_{2A}, A_{2B} and A₃ receptors, the latter being the most recently identified receptor.

The A₃ adenosine receptor (A₃AR) is involved in a variety of physiological processes. The receptor is highly expressed in various tumor cell types while expression in adjacent normal tissues is relatively low. Activation of the receptor by a specific synthetic agonist induces modulation of downstream signal transduction pathways which include the Wnt and the NF-κB, resulting in tumor growth inhibition (1-5).

In vivo studies have shown that A₃AR agonists inhibit the development of colon, prostate and pancreatic carcinomas as well as melanoma and hepatoma. A₃AR agonists were also been shown to act as anti-inflammatory agents by ameliorating the inflammatory process in different experimental autoimmune models such as rheumatoid arthritis, Crohn's disease and multiple sclerosis (6-10). It was proposed also that the A_{2A} and A₃ receptors mediate the anti-inflammatory effects of methotrexate (11).

A₃ adenosine receptor (A₃AR) expression levels are elevated in cancer cells as compared to normal cells (12). Thus, the A₃AR expression level has been described as a means for the diagnosis of cancer (13). In addition, A₃AR expression levels have also been described to be elevated in peripheral blood cells of patients with colorectal cancer (14).

Several members of the GPCR class of receptors have been reported to be modulated allosterically (15), i.e. these receptors have additional binding site(s) on a receptor that are distinct from the agonist binding site (orthosteric site, orthosterically modulated receptors), but that can modulate receptor activity.

Allosteric modulation of GPCRs has been characterized most extensively for muscarinic receptors (16), and it has been suggested that allosteric modulators may provide therapeutic advantages over orthosteric agonists. Such advantages may include greater subtype selectivity and fewer side effects (15).

The adenosine receptors are natural allosteric proteins because agonist-mediated signaling by GPCRs requires a conformational change in the receptor protein transmitted between two topographically distinct binding sites, one for the agonist and another for the G protein. Allosteric sites on GPCRs represent novel drug targets because allosteric modulators possess a number of advantages over classic orthosteric ligands, such as a ceiling level to the allosteric effect and a potential for greater GPCR subtype-selectivity.

Allosteric modulation of A₁ adenosine receptors was reported (17). A number of aminobenzoylthiophenes, including PD81723, were allosteric modulators of the A₁ adenosine receptor. These compounds were shown to be highly subtype-selective enhancers for A₁ adenosine receptors (17) and were less likely to cause desensitization and down-regulation of receptors than selective A₁ adenosine receptor agonists (18).

Some 1H-imidazo-[4,5-c]quinoline derivatives were described as selective allosteric enhancers of human A₃ adenosine receptors (19). Specifically, the derivatives were shown to influence the potency and maximal efficacy of agonist-induced responses while decreasing the dissociation of the agonist N⁶-(4-amino-3-[¹²⁵I]iodobenzyl)-5′-N-methylcarboxamido adeno sine from human A₃ adenosine receptors.

### SUMMARY OF THE INVENTION

The invention provides, in accordance with a first of its aspects, an A₃ adenosine receptor allosteric modulator (A₃RM), having the following general formula (I):

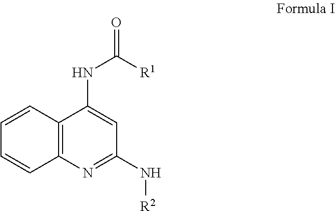

wherein:
   R₁ is a group selected from C₄-C₁₂ cycloalkyl, C₄-C₁₂ cycloallcenyl, C₆-C₁₂ aryl, C₄-C₁₂ heteroaryl, alkcycloalkyl, alkaryl, C₁-C₁₀ alkyl, C₂-C₁₀ alkenyl; C₂-C₁₀ allynyl, C₅-C₁₅ fused cycloalkyl, bicyclic aromatic or heteroaromatic ring; C₁-C₁₀ alkylether, amino, hydrazido, C₁-C₁₀ alkylamino, C₁-C₁₀ alkoxy, C₁-C₁₀-alkoxycarbony, C₁-C₁₀ alkanol, C₁-C₁₀ acyl, C₁-C₁₀ thioalkoxy, pyridylthio, thio, and C₁-C₁₀ alkylthio, acetoamido and sulfonic acid;
   R₂ is a group selected from hydrogen or a cyclic moiety selected from the group consisting of aryl, heteroary, alkaryl, alkheteroaryl, cycloalkyl, heterocycloalkyl, cycloalkenyl, heterocycloalkenyl, cycloalkynyl, heterocycloalkynyl, alkcycloalkyl, alkcycloheteroallyl, allccycloalkenyl and allcheterocycloalkenyl said cyclic moiety optionally substituted by at least one group selected from C₁-C₁₀ alkyl, halo, C₁-C₁₀ alkanol, hydroxyl, C₁-C₁₀ acyl, C₁-C₁₀ alkoxy; C₁-C₁₀-alkoxycarbony, C₁-C₁₀ alkoxylalkyl; C₁-C₁₀ thioalkoxy; C₁-C₁₀ allylether, amino, hydrazido, C₁-C₁₀ alkylamino, pyridylthio, C₂-C₁₀ alkenyl; C₂-C₁₀ alkynyl, thio, C₁-C₁₀ alkylthio, acetoamido and sulfonic acid; or said substituents can form together with an atom of a cyclic moiety a cycloalkyl, heterocycloalkyl, cycloalkenyl or heterocycloalkenyl fused to said cyclic moiety;
and pharmaceutically acceptable salts thereof, for use in the treatment of a condition which is treatable by adenosine or an adenosine agonist.

Specific, non-limiting A₃RM according to the invention include a 2,4-disubstituted quinoline derivative selected from:
N-(2-anilinoquinolin-4-yl)cyclopentanecarboxamide
N-{2-[(3,4-dichlorophenyl)amino]quinolin-4-yl}cyclopentanecarboxamide
N-[2-(benzylamino)quinolin-4-yl]cyclopentanecarboxamide
N-{2-[(4-methylphenyl)amino]quinolin-4-yl}cyclopentanecarboxamide
N-[2-(2,3-dihydro-1H-inden-5-ylamino)quinolin-4-yl]cyclopentanecarboxamide
N-{2-[(4-methoxyphenyl)amino]quinolin-4-yl}cyclopentanecarboxamide
N-{2-[(4-chlorophenyl)amino]quinolin-4-yl}cyclopentanecarboxamide
N-[2-(cyclopentylamino)quinolin-4-yl]cyclopentanecarboxamide
N-[2-(1H-indazol-6-ylamino)quinolin-4-yl]cyclopentanecarboxamide
N-(2-anilinoquinolin-4-yl)cyclohexanecarboxamide
N-{2-[(3,4-dichlorophenyl)amino]quinolin-4-yl}cyclohexanecarboxamide
N-{2-[(4-methylphenyl)amino]quinolin-4-yl}cyclohexanecarboxamide
N-[2-(2,3-dihydro-1H-inden-5-ylamino)quinolin-4-yl]cyclohexanecarboxamide
N-(2-anilinoquinolin-4-yl)benzamide
N-{2-[(3,4-dichloro-phenyl)amino]quinolin-4-yl}benzamide
N-(2-anilinoquinolin-4-yl)-2-furamide
N-{2-[(3,4-dichlorophenyl)amino]quinolin-4-yl}-2-furamide
N-(2-anilinoquinolin-4-yl)cyclobutanecarboxamide
N-{2-[(3,4-dichlorophenyl)amino]quinolin-4-yl}cyclobutanecarboxamide.

In one embodiment a 2,4-disubstituted quinoline derivative of the invention is N-{2-[(3,4-dichlorophenyl)amino]quinolin-4-yl}cyclohexanecarboxamide.

The invention also concerns an A₃RM for use in enhancing the activity of an A₃ adenosine receptor (A₃AR). In other words, a preferred embodiment of the invention concerns an A₃ adenosine receptor (A₃AR) enhancer.

Also the invention provides a method of altering/affecting an A₃ adenosine receptor (A₃AR) activity in a subject, the effect being similar to that obtained on said receptor by adenosine or an A₃AR agonist, the method comprises administering to said subject an amount of an A₃ adenosine receptor allosteric modulator (A₃RM), the amount being effective to modulate the A₃AR activity, wherein said A₃RM has the following general formula (I):

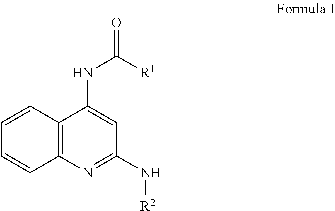

wherein:
   R₁ is a group selected from C₄-C₁₂ cycloalkyl, C₄-C₁₂ cycloalkenyl, C₆-C₁₂ aryl, C₄-C₁₂ heteroaryl, alkcycloalkyl, alkaryl, C₁-C₁₀ alkyl, C₂-C₁₀ alkenyl; C₂-C₁₀ alkynyl, C₅-C₁₅ fused cycloalkyl, bicyclic aromatic or heteroaromatic ring; C₁-C₁₀ alkylether, amino, hydrazido, C₁-C₁₀ alkylamino, C₁-C₁₀ alkoxy, C₁-C₁₀-alkoxycarbony, C₁-C₁₀ alkanol, C₁-C₁₀ acyl, C₁-C₁₀ thioalkoxy, pyridylthio, thio, and C₁-C₁₀ alkylthio, acetoamido and sulfonic acid;
   R₂ is a group selected from hydrogen or a cyclic moiety selected from the group consisting of aryl, heteroary, alkaryl, alkheteroaryl, cycloalkyl, heterocycloalkyl, cycloalkenyl, heterocycloalkenyl, cycloalkynyl, heterocycloalkynyl, alkcycloalkyl, alkcycloheteroalkyl, alkcycloalkenyl and alkheterocycloalkenyl said cyclic moiety optionally substituted by at least one group selected from C₁-C₁₀ alkyl, halo, C₁-C₁₀ alkanol, hydroxyl, C₁-C₁₀ acyl, C₁-C₁₀ alkoxy; C₁-C₁₀-alkoxycarbony, C₁-C₁₀ alkoxylalkyl; C₁-C₁₀ thioalkoxy; C₁-C₁₀ alkylether, amino, hydrazido, C₁-C₁₀ alkylamino, pyridylthio, C₂-C₁₀ alkenyl; C₂-C₁₀ alkynyl, thio, C₁-C₁₀ alkylthio, acetoamido and sulfonic acid; or said substituents can form together with an atom of a cyclic moiety a cycloalkyl, heterocycloalkyl, cycloalkenyl or heterocycloalkenyl fused to said cyclic moiety;
and pharmaceutically acceptable salts thereof.

Further provided is a method for treating a subject having a condition treatable by adenosine or an A₃AR agonist, the method comprising administering to said subject an amount of an A₃ adenosine receptor allosteric modulator (A₃RM), the amount being sufficient and effective to modulate (change, alter) the A₃AR activity, wherein said A₃RM has the following general formula (I):

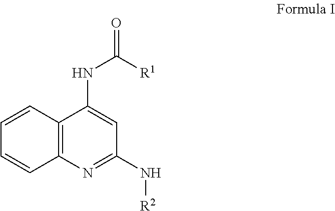

wherein:
   R₁ is a group selected from C₄-C₁₂ cycloalkyl, C₄-C₁₂ cycloalkenyl, C₆-C₁₂ aryl, C₄-C₁₂ heteroaryl, alkcycloalkyl, alkaryl, C₁-C₁₀ alkyl, C₂-C₁₀ alkenyl; C₂-C₁₀ alkynyl, C₅-C₁₅ fused cycloalkyl, bicyclic aromatic or heteroaromatic ring; C₁-C₁₀ alkylether, amino, hydrazido, C₁-C₁₀ alkylamino, C₁-C₁₀ alkoxy, C₁-C₁₀-alkoxycarbony, alkanol, acyl, C₁-C₁₀ thioalkoxy, pyridylthio, thio, and C₁-C₁₀ alkylthio, acetoamido and sulfonic acid;
   R₂ is a group selected from hydrogen or a cyclic moiety selected from the group consisting of aryl, heteroary, alkaryl, alkheteroaryl, cycloalkyl, heterocycloalkyl, cycloalkenyl, heterocycloalkenyl, cycloalkynyl, heterocycloalkynyl, alkcycloalkyl, alkcycloheteroalkyl, alkcycloalkenyl and alkheterocycloalkenyl said cyclic moiety optionally substituted by at least one group selected from C₁-C₁₀ alkyl, halo, C₁-C₁₀ alkanol, hydroxyl, C₁-C₁₀ acyl, C₁-C₁₀ alkoxy; C₁-C₁₀-alkoxycarbony, C₁-C₁₀ alkoxylalkyl; C₁-C₁₀ thioalkoxy; C₁-C₁₀ alkylether, amino, hydrazido, C₁-C₁₀ alkylamino, pyridylthio, C₂-C₁₀ alkenyl; C₂-C₁₀ alkynyl, thio, C₁-C₁₀ alkylthio, acetoamido and sulfonic acid; or said substituents can form together with an atom of a cyclic moiety a cycloalkyl, heterocycloalkyl, cycloalkenyl or heterocycloalkenyl fused to said cyclic moiety;
and pharmaceutically acceptable salts thereof.

Also provided by the invention is a pharmaceutical composition comprising as active ingredient an A₃RM as defined herein or a 2,4-disubstituted quinoline derivative as provided hereinabove. The pharmaceutical composition is, in accordance with one embodiment, in a form suitable for oral administration.

The invention also provides the use of an A₃RM as defined herein for the preparation of a pharmaceutical composition for the treatment of a condition treatable with adenosine or an A₃AR agonist.

Finally, provided by the invention is a kit comprising an A₃RM as defined herein and instructions for use of said A₃RM in treatment of a condition in a subject which is treatable by adenosine or an A₃AR agonist.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a scheme of the synthetic procedure of 2,4-disubstituted quinoline derivatives (compounds 16-20).

FIG. 2 is a scheme of the synthetic procedure of 2,4-disubstituted quinoline derivatives (compounds 21-34).

### DETAILED DESCRIPTION OF EXEMPLARY EMBODIMENTS

The present invention concerns allosteric modulation (inhibition or enhancement, albeit mostly enhancement) of the A₃ adenosine receptor (A₃AR) by use of 2,4-disubstituted quinoline derivatives. Specifically, the invention is based on the finding that 2,4-disubstituted quinoline derivatives can effectively increase the efficacy of the A₃ adenosine receptor, upon binding thereto.

As appreciated, while the invention is described in the following detailed description with reference to A₃ adenosine receptor modulators for use in treatment, it is to be understood that also encompassed within the present invention are pharmaceutical compositions comprising an A₃ adenosine receptor allosteric modulator, methods making use of such A₃ adenosine receptor allosteric modulators; kits comprising an A₃ adenosine receptor allosteric modulator and instructions for use of the same as well as some novel 2,4-disubstituted quinoline derivatives found to be specifically effective as allosteric modulators, preferably enhancers, of the receptor.

As used herein, the term “allosteric modulation” which may be used interchangeably with the term “allosteric regulation” denotes the alteration or change (either increase or decrease) in the activity of an enzyme, receptor or other protein by binding of an effector molecule at the A₃ adenosine receptor (A₃AR) allosteric site which is different from the binding site of the endogenous ligand of this A₃AR, the latter being defined as the orthosteric binding site.

Effector molecules that enhance the said activity by binding to the A₃AR allosteric site are referred to herein as “allosteric activators” or “allosteric enhancers”, whereas those that decrease the activity are called “allosteric inhibitors”.

Thus, in accordance with a first of its aspects, the present invention provides an A₃ adenosine receptor allosteric modulator (A₃RM) for use in the treatment of a condition which requires for its treatment modulation of an A₃ adenosine receptor (A3AR), and that is treatable with adenosine or an A₃ adenosine receptor (A₃AR) agonist, wherein the A₃RM has the following general formula (I):

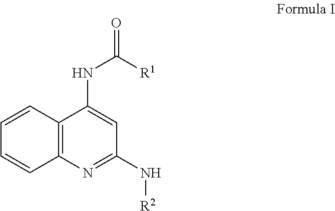

wherein:
   R₁ is a group selected from C₄-C₁₂ cycloalkyl, C₄-C₁₂ cycloalkenyl, C₆-C₁₂ aryl, C₄-C₁₂ heteroaryl, alkcycloalkyl, alkaryl, C₁-C₁₀ alkyl, C₂-C₁₀ alkenyl; C₂-C₁₀ alkynyl, C₅-C₁₅ fused cycloalkyl, bicyclic aromatic or heteroaromatic ring; C₁-C₁₀ alkylether, amino, hydrazido, C₁-C₁₀ alkylamino, C₁-C₁₀ alkoxy, C₁-C₁₀-alkoxycarbony, C₁-C₁₀ alkanol, C₁-C₁₀ acyl, C₁-C₁₀ thioalkoxy, pyridylthio, thio, and C₁-C₁₀ alkylthio, acetoamido and sulfonic acid;
   R₂ is a group selected from hydrogen or a cyclic moiety selected from the group consisting of aryl, heteroary, alkaryl, alkheteroaryl, cycloalkyl, heterocycloalkyl, cycloalkenyl, heterocycloalkenyl, cycloalkynyl, heterocycloalkynyl, alkcycloalkyl, alkcycloheteroalkyl, alkcycloalkenyl and alkheterocycloalkenyl said cyclic moiety optionally substituted by at least one group selected from C₁-C₁₀ alkyl, halo, C₁-C₁₀ alkanol, hydroxyl, C₁-C₁₀ acyl, C₁-C₁₀ alkoxy; C₁-C₁₀-alkoxycarbony, C₁-C₁₀ alkoxylalkyl; C₁-C₁₀ thioalkoxy; C₁-C₁₀ alkylether, amino, hydrazido, C₁-C₁₀ alkylamino, pyridylthio, C₂-C₁₀ alkenyl; C₂-C₁₀ alkynyl, thio, C₁-C₁₀ alkylthio, acetoamido and sulfonic acid; or said substituents can form together with an atom of a cyclic moiety a cycloalkyl, heterocycloalkyl, cycloalkenyl or heterocycloalkenyl fused to said cyclic moiety;

and pharmaceutically acceptable salts thereof, for use in the treatment of a condition which is treatable by adenosine or an adenosine agonist.

The term “alkyl” is used herein to refer to a linear or branched hydrocarbon chain having from 1 to 10 carbon atoms and more preferably 1 to 6 carbon atoms including, but not limited to, methyl, ethyl, n-propyl, isopropyl, n-butyl, t-butyl, n-heptyl, octyl and the like.

Similarly, the terms “alkenyl” and “alkynyl” denote a linear or branched hydrocarbon chain having, respectively, from 2 to 10, or from 3 to 10 carbon atoms and more preferably 2 to 6 or 3 to 6 carbon atoms, the alkenyl or alkynyl having at least one unsaturated bond.

The alkyl, alkenyl or alkynyl substituents may be substituted with a heteroatom containing group. Thus, it should be understood that while not explicitly stated, any of the alkyl modifications defined hereinabove and below, such as alkylthio, alkoxy, akanol, alkylamine etc, also include the corresponding alkenyl or alkynyl modifications, such as, akenylthio, akenyloxy, alkenol, alkenylamine, or respectively, akynylthio, alkynyloxy, alkynol, alkynylamine.

The term “aryl” denotes an unsaturated aromatic carbocyclic group of from 5 to 14 carbon atoms having a single ring (e.g., phenyl) or multiple condensed rings (e.g., naphthyl or anthryl). Preferred aryls include phenyl, indanyl, benzimidazole.

The term “alkaryl” refers to -alkylene-aryl groups preferably having from 1 to 10 carbon atoms in the alkylene moiety and from 6 to 14 carbon atoms in the aryl moiety. Such alkaryl groups are exemplified by benzyl, phenethyl and the like.

The term “substituted aryl” refers to an aromatic moiety which is substituted with from 1 to 3 substituents as defined above. A variety of substituents are possible, as appreciated by those versed in the art. Nonetheless, some preferred substituents include, without being limited thereto, halogen, (substituted) amino, nitro, cyano, alkyl, alkoxy, acyloxy or alkanol, sulphonyl, sulphynyl.

The term “halo” or “halogen” refers to fluoro, chloro, bromo and iodo, preferably to chloro.

The term “acyl” refers to the groups H—C(O)— as well as alkyl-C(O)—.

The term “alkanol” refers to the group —COH as well as alk-OH, “alk” denoting an alkylene, alkenylene or alkynylene chain.

The term “alkoxy” is used herein to mean —O-alkyl, including, but not limited to, methoxy, ethoxy, propoxy, isopropoxy, n-butoxy and the like.

The term “alkylthio” is used herein to mean —S-alkyl, including, but not limited to, methylthio, ethylthio, n-propylthio, isopropylthio, n-butylthio and the like.

The term “alkoxyalkyl” is used herein to mean -alkyl-O-alkyl, including, but not limited to, methoxymethyl, ethoxymethyl, n-propoxymethyl, isopropoxymethyl, n-butoxymethyl, isobutoxymethyl, t-butoxymethyl and the like.

The term “cycloalkyl” is used herein to mean cyclic hydrocarbon radicals including, but not limited to, cyclopropyl, cyclobutyl, cyclopentyl, cyclohexyl, cycloheptyl and the like.

The term “alkoxycarbonyl” is used herein to mean —C(O)O-alkyl, including, but not limited to, methoxycarbonyl, ethoxycarbonyl, propoxycarbonyl and the like.

According to one embodiment of the invention R¹ is represents a cycloalkyl, aryl or heteroaryl.

In one embodiment R² is selected from aryl, alkaryl, cycloalkyl, the aryl or cycloalkyl being optionally substituted by at least one substituent selected from C₁-C₁₀ alkyl, halo (preferably chloro) and C₁-C₁₀ alkylether.

In another embodiment R¹ is selected from C₄-C₆ cycloalkyl, phenyl or a five membered heterocyclic aromatic ring having the following formula (II):

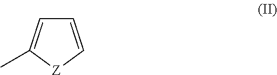

wherein Z is selected from O, S or NH; and

R² is selected from C₄-C₆ cycloalkyl, phenyl, alkphenyl, or an aromatic ring fused to a five membered cyclic or heteroaromatic ring having the following formulae (IIIa) or (IIIb):

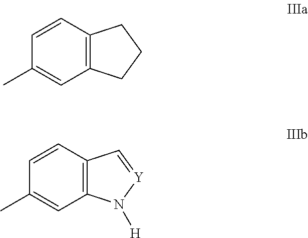

wherein Y is selected from N or CH.

the aryl or cycloalkyl ring in said cycloalkyl, phenyl, alkphenyl or in formulae (Va) or (Vb) being optionally substituted with a substituent selected from C₁-C₁₀ alkyl, halo, or C₁-C₁₀ alkylether.

In yet another embodiment, R¹ is selected from C₄-C₆ cycloalkyl, phenyl or a five membered heterocyclic aromatic ring having the following formula (IIa)

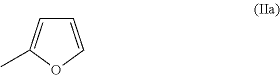

R² is selected from cyclopentyl, phenyl, methylphenyl, or an aromatic ring fused to a five membered cyclic or hetero aromatic ring having the following formulae (IIIa) or (IIIb):

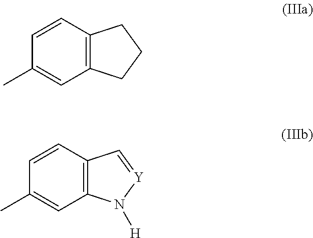

the phenyl being optionally substituted once or more with a methyl, chloro or methylether.

R² may also be represented by the general formula (IV):

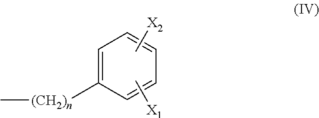

wherein n is 0 or an integer selected from 1-5; preferably, n is 0, 1 or 2; and
X₁ and X₂ which may be the same or different, are selected from hydrogen, halogen, alkyl, alkanol or alkoxy, indanyl, pyrroline provided that when said n is 0, X₁ and X₂ are not hydrogen.

Specific, albeit non-limiting A3RM according to the invention, include the following 2,4-disubstituted quinoline derivatives (in brackets their number according to the following Table 1):
N-(2-anilinoquinolin-4-yl)cyclopentanecarboxamide (16)
N-{2-[(3,4-dichlorophenyl)amino]quinolin-4-yl}cyclopentanecarboxamide (17)
N-[2-(benzylamino)quinolin-4-yl]cyclopentanecarboxamide (18)
N-{2-[(4-methylphenyl)amino]quinolin-4-yl}cyclopentanecarboxamide (19)
N-[2-(2,3-dihydro-1H-inden-5-ylamino)quinolin-4-yl]cyclopentanecarboxamide (20)
N-{2-[(4-methoxyphenyl)amino]quinolin-4-yl}cyclopentanecarboxamide (21)
N-{2-[(4-chlorophenyl)amino]quinolin-4-yl}cyclopentanecarboxamide (22)
N-[2-(cyclopentylamino)quinolin-4-yl]cyclopentanecarboxamide (23)
N-[2-(1H-indazol-6-ylamino)quinolin-4-yl]cyclopentanecarboxamide (24)
N-(2-anilinoquinolin-4-yl)cyclohexanecarboxamide (25)
N-{2-[(3,4-dichlorophenyl)amino]quinolin-4-yl}cyclohexanecarboxamide (26)
N-{2-[(4-methylphenyl)amino]quinolin-4-yl}cyclohexanecarboxamide (27).
N-[2-(2,3-dihydro-1H-inden-5-ylamino)quinolin-4-yl]cyclohexanecarboxamide (28)
N-(2-anilinoquinolin-4-yl)benzamide (29)
N-{2-[(3,4-dichloro-phenyl)amino]quinolin-4-yl}benzamide (30)
N-(2-anilinoquinolin-4-yl)-2-furamide (31)
N-{2-[(3,4-dichlorophenyl)amino]quinolin-4-yl}-2-furamide (32)
N-(2-anilinoquinolin-4-yl)cyclobutanecarboxamide (33)
N-{2-[(3,4-dichlorophenyl)amino]quinolin-4-yl}cyclobutanecarboxamide (34).

In one embodiment, said A₃RM is an A₃ adenosine receptor allosteric enhancer, i.e. for use in enhancing the activity of an A₃ adenosine receptor (A₃AR). In accordance with this embodiment, the A₃RM has the above identified formula (I), wherein said R¹ is a C₄-C₆ cycloalkyl or a phenyl; and R² is selected from C₄-C₆ cycloalkyl, phenyl or an aromatic ring fused to a five membered cycloalkyl having the following formulae

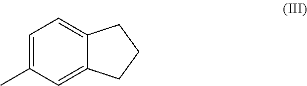

the phenyl moiety in R² being unsubstituted or substituted at least once with a C₁-C₃ alkyl, halogen or C₁-C₃ alkether.

In accordance with a more particular embodiment of the A3AR enhancer, the R¹ is selected from cyclopentyl, cyclohexyl, cyclo butyl or phenyl; and the is selected from cyclopentyl, phenyl or an aromatic ring fused to a five membered cycloalkyl having the following formulae (III):

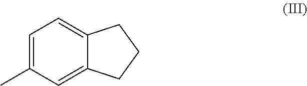

the phenyl moiety in R² being unsubstituted or substituted at least once with a methyl, Cl or methylether.

A non-limiting list of A3AR enhancers include the following 2,4-disubstituted quinoline derivatives:
N-{2-[(3,4-dichlorophenyl)amino]quinolin-4-yl}cyclopentanecarboxamide
N-{2-[(4-methoxyphenyl)amino]quinolin-4-yl}cyclopentanecarboxamide
N-{2-[(4-chlorophenyl)amino]quinolin-4-yl}cyclopentanecarboxamide
N-(2-anilinoquinolin-4-yl)cyclohexanecarboxamide
N-{2-[(3,4-dichlorophenyl)amino]quinolin-4-yl}cyclohexanecarboxamide
N-{2-[(4-methylphenyl)amino]quinolin-4-yl}cyclohexanecarboxamide
N-[2-(2,3-dihydro-1H-inden-5-ylamino)quinolin-4-yl]cyclohexanecarboxamide
N-{2-[(3,4-dichlorophenyl)amino]quinolin-4-yl}cyclobutanecarboxamide.

A more specific group of 2,4-disubstituted quinoline derivatives include, without being limited thereto:
N-{2-[(4-chlorophenyl)amino]quinolin-4-yl}cyclopentanecarboxamide
N-(2-anilinoquinolin-4-yl)cyclohexanecarboxamide
N-{2-[(3,4-dichlorophenyl)amino]quinolin-4-yl}cyclohexanecarboxamide
N-[2-(2,3-dihydro-1H-inden-5-ylamino)quinolin-4-yl]cyclohexanecarboxamide.

A preferred A₃AR enhancer is N-{2-[(3,4-dichlorophenyl)amino]quinolin-4-yl}cyclohexanecarboxamide.

With respect to the enhancing activity of the A3RM, the enhancement is also defined by the occurrence of one or more of the following:
an increase of at least 15% in efficacy of said A₃AR by binding of said A3AR enhancer to an allosteric site of said A₃AR; or
a decrease in dissociation rate of adenosine or an A₃AR agonist from its binding site;
less than 60% of displacement of adenosine or an A₃AR agonist from its binding site.

Further, when referring to modulation by enhancement of the activity of the receptor, the condition treatable by adenosine or an A3AR agonist, and to be treated by said allosteric enhancer comprises, without being limited thereto, a malignancy, an immuno-compromised affliction, high intraocular pressure or a condition associated with high intraocular pressure. To this end, the subject requiring said treatment may also be treated in combination with an agonist to the orthosteric binding site of said A₃R.

Conditions for which the A3AR allosteric enhancer is to be used include, rheumatoid arthritis (RA), glaucoma or for enhancing a subject's myeloid system.

The invention also concerns a method of affecting an A₃ adenosine receptor (A₃AR) activity in a subject, the effect being similar to that of adenosine or an A₃AR agonist on said receptor, the method comprises administering to said subject an amount of an A₃ adenosine receptor allosteric modulator (A₃RM), the amount being effective to modulate the A₃AR activity, wherein said A₃RM has the general formula (I) as defined above.

When referring to an effect being similar to that of adenosine or an A₃AR agonist on said receptor it is meant that if adenosine and/or an A₃AR agonist increase the activity of an enzyme, protein etc. by binding to the receptor, a similar effect by the A₃RM would be also an increase in the activity of said enzyme, protein etc. The change in activity should be to an extent that a therapeutic effect is achieved by the binding of the A₃RM, the therapeutic effect being defined below with respect to treatment with A₃RM.

Further provided is a method for treating a subject having a condition treatable by adenosine or an A₃AR agonist, the method comprising administering to said subject an amount of an A₃ adenosine receptor allosteric modulator (A₃RM), the amount being effective to modulate the A₃AR activity, wherein said A₃RM has the general formula (I) as defined above.

The term treatment “treatment” as used herein refers to the therapeutic effect achieved by the administering of an amount of an A₃AM according to the invention and specifically the substituted quinoline derivatives defined herein, the therapeutic effect, being selected from one or more of the following: amelioration of undesired symptoms associated with condition treatable with adenosine or an A₃ adenosine receptor agonist (A₃AR agonist), prevention of the manifestation of such symptoms before they occur, slowing down a progression of the condition, slowing down any deterioration of symptoms of the condition, enhancement of onset of a remission period of a condition, slowing down of any irreversible damage caused in a progressive chronic stage of the condition, delaying of the onset of said progressive stage, lessening of the severity or cure of the condition, improving survival rate or more rapid recovery from the condition, preventing the condition form occurring or a combination of two or more of the above.

A variety of conditions may be treated by the modulation of the A₃AR depending on the specific effect the 2,4-disubstituted quinoline has on the receptor, i.e. inhibition or enhancement.

When modulation comprises inhibition of or decrease in efficacy of the receptor, the condition may be any condition treatable by the binding of an A₃ adenosine receptor antagonist. Such conditions comprise, without being limited thereto, certain malignancies or certain immuno-compromised afflictions.

When modulation comprises enhancement or increase in efficacy of the receptor, the condition may be any condition which is treatable by the binding of adenosine or an A₃ adenosine receptor agonist. Such conditions comprise, without being limited thereto, hyperproliferative disorders, and in particular all types of solid tumors; skin proliferative diseases (e.g. psoriasis); a variety of benign hyperplasic disorders; inflammatory diseases; ischemic conditions, such as myocardial or renal ischemia and conditions associated with intraocular pressure (e.g. glaucoma).

The term “solid tumors” refers to carcinomas, sarcomas, adenomas, and cancers of neuronal origin and if fact to any type of cancer which does not originate from the hematopoeitic cells and in particular concerns: carcinoma, sarcoma, adenoma, hepatocellular carcinoma, hepatocellularcarcinoma, hepatoblastoma, rhabdomyosarcoma, esophageal carcinoma, thyroid carcinoma, ganglioblastoma, fibrosarcoma, myxosarcoma, liposarcoma, cohndrosarcoma, osteogenic sarcoma, chordoma, angiosarcoma, endotheliosarcoma, lymphagiosarcoma, synovioama, Ewing's tumor, leimyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocyoma, medulloblastoma, craniopharyngioma, ependynoma, pinealoma, retinoblastoma, multiple myeloma, rectal carcinoma, thyroid cancer, head and neck cancer, brain cancer, cancer of the peripherial nervous system, cancer of the central nervous system, neuroblastoma, cancer of the endometrium, as well as metastasis of all the above. It has been shown in accordance with the invention that increased expression of A₃AR can be found not only in the primary tumor site but also in metastases thereof.

Benign hyperplasic disorders include, without being limited thereto, benign prostate hyperplasia (BPH), non-tumorigenic polyps in the digestive tract, in the uterus and others.

Inflammatory diseases include, without being limited thereto, rheumatoid arthritis, Crohn's disease, multiple sclerosis and others.

When referring to treatment of a condition heatable by adenosine or an A₃AR agonist, the A₃R enhancer according to the invention is preferably 2,4-disubstituted quinoline derivative selected from:
N-{2-[(3,4-dichlorophenyl)amino]quinolin-4-yl}cyclopentanecarboxamide
N-{2-[(4-methoxyphenyl)amino]quinolin-4-yl}cyclopentanecarboxamide
N-{2-[(4-chlorophenyl)amino]quinolin-4-yl}cyclopentanecarboxamide
N-(2-anilinoquinolin-4-yl)cyclohexanecarboxamide
N-{2-[(3,4-dichlorophenyl)amino]quinolin-4-yl}cyclohexanecarboxamide
N-{2-[(4-methylphenyl)amino]quinolin-4-yl}cyclohexanecarboxamide
N-[2-(2,3-dihydro-1H-inden-5-ylamino)quinolin-4-yl]cyclohexanecarboxamide
N-{2-[(3,4-dichlorophenyl)amino]quinolin-4-yl}cyclobutanecarboxamide.

More preferably, the A₃R enhancer is selected from:
N-{2-[(4-chlorophenyl)amino]quinolin-4-yl}cyclopentanecarboxamide
N-(2-anilinoquinolin-4-yl)cyclohexanecarboxamide
N-{2-[(3,4-dichlorophenyl)amino]quinolin-4-yl}cyclohexanecarboxamide
N-[2-(2,3-dihydro-1H-inden-5-ylamino)quinolin-4-yl]cyclohexanecarboxamide.

According to the invention, the A₃RM may be administered in combination with a ligand to the orthosteric binding site. When modulation involves enhancement of the receptor, the A₃RM may be administered in combination with adenosine or an A₃AR agonist; when modulation involved inhibition of the receptor, the A₃RM may be administered in combination with an A₃AR antagonist.

The term “combination” includes a schedule of treatment that involves administration of at least the A₃RM and the ligand to the orthosteric site. The schedule of treatment may comprise simultaneous or co-administration of the A₃RM and the ligand, or with an interval between administrations. The A₃RM and the ligand may be formulated together or may be included in two different formulations. In addition, the mode of administration and/or the schedule of treatment (i.e. doses per time period) of the A₃RM and the ligand may be different.

According to an embodiment of the present invention, the A₃RM is administered to the subject orally; although other administration routes are applicable, including parenteral (intravenous, intramuscular, intra-arterial, subcutaneous, intranasal, via the lungs (inhalation)).

The invention also provides novel 2,4-disubstituted quinoline derivative selected from:
N-(2-anilinoquinolin-4-yl)cyclopentanecarboxamide
N-{2-[(3,4-dichlorophenyl)amino]quinolin-4-yl}cyclopentanecarboxamide
N-[2-(benzylamino)quinolin-4-yl]cyclopentanecarboxamide
N-{2-[(4-methylphenyl)amino]quinolin-4-yl}cyclopentanecarboxamide
N-[2-(2,3-dihydro-1H-inden-5-ylamino)quinolin-4-yl]cyclopentanecarboxamide
N-{2-[(4-methoxyphenyl)amino]quinolin-4-yl}cyclopentanecarboxamide
N-{2-[(4-chlorophenyl)amino]quinolin-4-yl}cyclopentanecarboxamide
N-[2-(cyclopentylamino)quinolin-4-yl]cyclopentanecarboxamide
N-[2-(1H-indazol-6-ylamino)quinolin-4-yl]cyclopentanecarboxamide
N-(2-anilinoquinolin-4-yl)cyclohexanecarboxamide
N-{2-[(3,4-dichlorophenyl)amino]quinolin-4-yl}cyclohexanecarboxamide
N-{2-[(4-methylphenyl)amino]quinolin-4-yl}cyclohexanecarboxamide
N-[2-(2,3-dihydro-1H-inden-5-ylamino)quinolin-4-yl]cyclohexanecarboxamide
N-(2-anilinoquinolin-4-yl)benzamide
N-{2-[(3,4-dichloro-phenyl)amino]quinolin-4-yl}benzamide
N-(2-anilinoquinolin-4-yl)-2-furamide
N-{2-[(3,4-dichlorophenyl)amino]quinolin-4-yl}-2-furamide
N-(2-anilinoquinolin-4-yl)cyclobutanecarboxamide
N-{2-[(3,4-dichlorophenyl)amino]quinolin-4-yl}cyclobutanecarboxamide.

Specifically, the invention provides novel 2,4-disubstituted quinoline derivatives selected from:
N-{2-[(3,4-dichlorophenyl)amino]quinolin-4-yl}cyclopentanecarboxamide
N-{2-[(4-methoxyphenyl)amino]quinolin-4-yl}cyclopentanecarboxamide
N-{2-[(4-chlorophenyl)amino]quinolin-4-yl}cyclopentanecarboxamide
N-(2-anilinoquinolin-4-yl)cyclohexanecarboxamide
N-{2-[(3,4-dichlorophenyl)amino]quinolin-4-yl}cyclohexanecarboxamide
N-{2-[(4-methylphenyl)amino]quinolin-4-yl}cyclohexanecarboxamide
N-[2-(2,3-dihydro-1H-inden-5-ylamino)quinolin-4-yl]cyclohexanecarboxamide
N-{2-[3,4-dichlorophenyl)amino]quinolin-4-yl}cyclobutanecarboxamide.

More specifically, the invention provides novel 2,4-disubstituted quinoline derivatives selected from:
N-{2-[(4-chlorophenyl)amino]quinolin-4-yl}cyclopentanecarboxamide
N-(2-anilinoquinolin-4-yl)cyclohexanecarboxamide
N-{2-[(3,4-dichlorophenyl)amino]quinolin-4-yl}cyclohexanecarboxamide
N-[2-(2,3-dihydro-1H-inden-5-ylamino)quinolin-4-yl]cyclohexanecarboxamide.

A preferred novel 2,4-disubstituted quinoline derivative according to the invention is N-{2-[(3,4-dichlorophenyl)amino]quinolin-4-yl}cyclohexanecarboxamide.

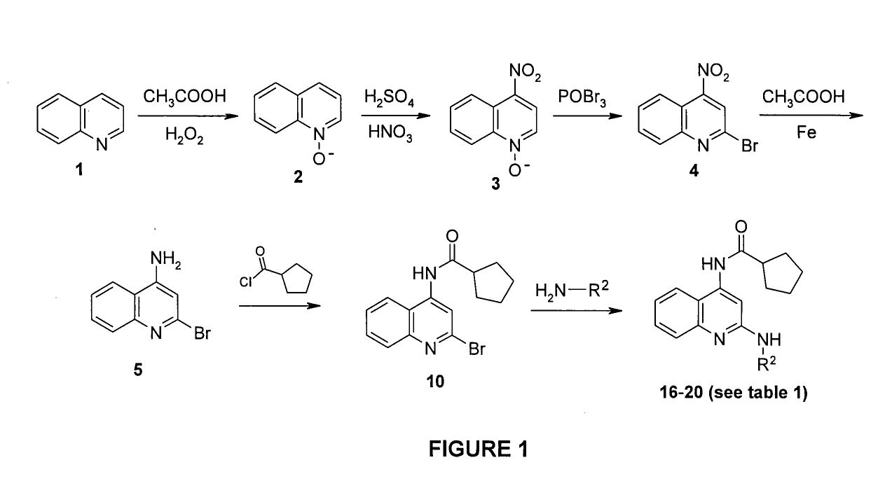

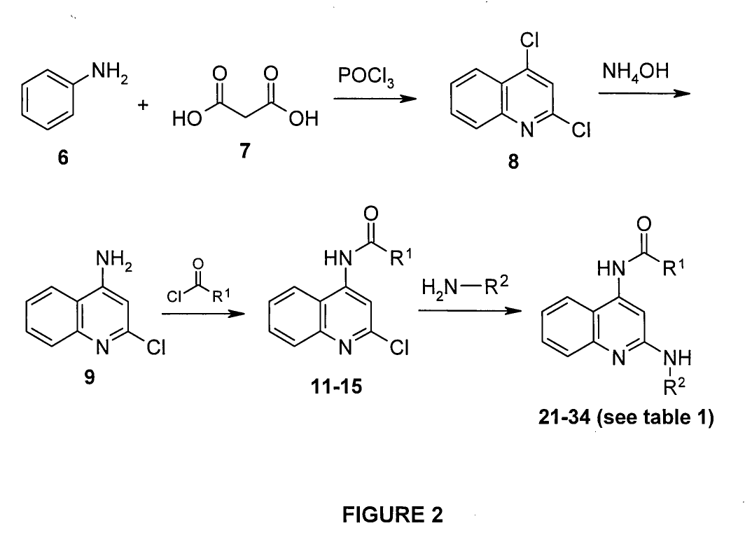

In general, the novel derivatives 16-20 were synthesized as shown in the Scheme depicted in FIG. 1, while the other novel derivatives (21-34) were synthesized as shown in the Scheme depicted in FIG. 2. Oxidation of quinoline resulted in quinoline-1-oxide (2) [Ochiai, E. Recent Japanese work on the chemistry of pyridine 1-oxide and related compounds, J. Org. Chem. 1953, 18, 534-551; Zhong, P. et al. A simple and efficient method for the preparation of heterocyclic N-oxide, Synth. Commun. 2004, 34, 247-253], which was nitrated to give 4-nitroquinoline-1-oxide (3) [Taylor Jr, E. C. et al. 3-Methyl-4-nitropyridine-1-oxide. Org. Synth. 1963. Coll. Vol. 4. 654-656; Yokoyama, A. et al. Nitration of quinoline 1-oxide: mechanism of regioselectivity, Chem. Pharm. Bull. 1997, 45, 279-283]. 4-Nitroquinoline-1-oxide (3) was treated with phosphorous oxybromide to afford 2-bromo-4-nitroquinoline (4) [Hamana, M. et al. A new deoxidation reaction of aromatic tertiary amine oxides. Reaction of 4-nitroquinoline 1-oxide with phosphorus bromide. Chem. Abstr. 1957. 51. 6639; Woźniak, M. et al. Amination of 4-nitroquinoline with liquid methylamine/potassium permanganate, Chem. Heterocyc. Comp. 1998, 34, 837-840]. This was converted into 4-amino-2-bromoquinoline (5) with iron powder in acetic acid [Komblum, N. et al. The reduction of optically active 2-nitrooctane and α-phenylnitroethane. J. Am. Chem. Soc. 1955. 77. 6266-6269; Den Hertog, H. J. et al. Reactivity of aminobromoquinolines towards potassium amide in liquid ammonia, Rec. Tray. Chim. des Pays-Bas. 1972, 91, 841-849]. Ring-closure reaction of malonic acid with aniline in phosphorus oxychloride gave 2,4-dichloroquinoline (8) [Osborne, A. G. et al. 2,4-dihalogenoquinolines. Synthesis, orientation effects and ¹H and ¹³C NMR spectral studies, J. Chem. Soc. Perkin Trans. I. 1993, 1, 2747-2755], which was subsequently treated with ammonia in the microwave to give 2-amino-4-chloroquinoline (9) [von Büchi, J. et al. Die tuberkulostatische wirkung von 2-oxy-4-amino-chinolin-derivaten, Helv. Chim. Acta. 1949, 32, 1806-1814; Wojahn, H. Untersuchungen über den zusammenhang von chemischer constitution and anästhesierender wirkung bei 2-alkoxy-chinolin-derivaten, Arch. Pharm. 1936, 274, 83-106]. Reaction of compounds 5 and 9 with carbonyl chlorides [Chang, L. C. W. et al. 2,4,6-Trisubstituted pyrimidines as a new class of selective adenosine A₁ receptor antagonists, J. Med. Chem. 2004, 47, 6529-6540] and then subsequently with the appropriate amines afforded the desired compounds 16-20 and 21-34, respectively [Göblyös, A. et al. Structure-activity relationships of new 1H-imidazo[4,5-c]quinolin-4-amine derivatives as allosteric enhancers of the A₃ adenosine receptor, J. Med. Chem. 2006, 49, 3354-3361].

It has now been found that modifying the von Büchi or Wojahn procedure, noted above, by using microwave irradiation (a period of about 2.5 hours max) resulted in an easier and more straightforward purification of the end products. This was unexpected since microwave irradiation has not been considered relevant for this procedure.

It is noted that the above specific 2,4-disubstituted quinoline derivatives are novel per se and most have been shown to enhance the response obtained by their allosteric binding to A₃AR.

The 2,4-disubstituted quinoline derivatives of the invention were shown to have, on the one hand, reduced affinity, if any, to the orthosteric binding sites of the A₁, A_{ZA}, and A_{2B} adenosine receptors (not shown) and reduced affinity to the orthosteric binding site of the A₃ adenosine receptor (column 4 in Table 2), and on the other hand, high efficacy at the allosteric site of the A₃ adenosine receptor (last column of Table 2). The selective affinity/efficacy of the derivatives disclosed herein is particularly evident with respect to compounds 22, 25, 26, and 28 in Table 2. These four compounds show little (<50%) displacement of orthosteric ligand binding (column 4 in Table 2), whereas they have obvious enhancing activity (up to 249% compared to a control value of 100%—see last column in Table 2).

As further shown in Table 2 hereinafter, the specific 2,4-disubstituted quinoline derivatives of the invention were shown to increase the activity of the A₃AR. Thus, as indicated above a preferred embodiment of the invention comprises enhancement of A₃AR activity.

Thus, when referring to the substituted quinoline derivatives of formula (I) and the specific novel 2,4-disubstituted quinoline derivatives of the invention, and in line with the above definition of allosteric enhancer, the effect of the substituted quinoline derivatives on the receptor is exhibited by an increase of at least 15% in the efficacy of the A₃ adenosine receptor by binding of the substituted quinoline to the allosteric site of the receptor, which was measured as a decrease (of at least 30%, preferably 40%) in dissociation rate of an A₃AR agonist to the orthosteric binding site.

The invention also provides a pharmaceutical composition comprising as active ingredient a novel 2,4-disubstituted quinoline derivative as provided herein above and below.

Further provided by the invention is a pharmaceutical composition for treating a condition which is treatable with adenosine or an A₃AR agonist, comprising as active ingredient an A₃RM having the formula (I) as defined herein.

The composition of the invention may comprise a combination of A₃RM and a ligand to the orthosteric binding site of said A₃R. In one embodiment said ligand is an A₃R agonist and said composition comprises an A₃ adenosine receptor allosteric enhancer.

In one embodiment, the A₃AM in the composition for treating a condition treatable by adenosine is a 2,4-disubstituted quinoline derivative as disclosed herein.

In one embodiment, the pharmaceutical composition of the invention is in a form suitable for oral administration.

The invention further provides a use of the A₃RM having the following general formula (I) and pharmaceutically acceptable salts thereof; for the preparation of a pharmaceutical composition for treatment of a condition which is treatable by adenosine or an A₃AR agonist.

In making the compositions of this invention, the substituted quinoline derivative of formula (I) or the novel 2,4-disubstituted quinoline derivative is usually mixed with the excipient, diluted by an excipient or enclosed within a carrier which can be in the form of a capsule, sachet, paper or other container. The term “physiologically acceptable excipient” denotes any excipient that is useful in preparing a pharmaceutical composition or formulation that is generally safe, non-toxic and neither biologically nor otherwise undesirable, and includes an excipient that is acceptable for veterinary use as well as human pharmaceutical use. When the excipient serves as a diluent, it can be a solid, semi-solid, or liquid material, which acts as a vehicle, carrier or medium for the 2,4-disubstituted quinoline derivative. Thus, the compositions can be in the form of tablets, pills, powders, lozenges, sachets, cachets, elixirs, suspensions, emulsions, solutions, syrups, aerosols (as a solid or in a liquid medium), soft and hard gelatin capsules, suppositories, sterile injectable solutions, and sterile packaged powders.

The effective amount of substituted quinoline derivative of formula (I) or the novel 2,4-disubstituted quinoline derivatives of the invention in the pharmaceutical composition may vary or be adjusted depending upon the particular application, the manner or introduction, the potency of the particular compound, and the desired concentration. The effective amount is typically determined in appropriately designed clinical trials (dose range studies) and the person versed in the art will know how to properly conduct such trials in order to determine the effective amount. As generally known, an effective amount depends on a variety of factors including the affinity of the 2,4-disubstituted quinoline derivative to the allosteric binding site, its distribution profile within the body, a variety of pharmacological parameters such as half life in the body, on undesired side effects, if any, on factors such as age and gender, etc.

The A₃RM is typically administered in unit dosage forms. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. The amount of therapeutically active compound in such a unit dosage form may vary from about 0.5 mg to 500 mg.

In this case, the composition of the invention will typically be administered over an extended period of time in a single daily dose, in several doses a day, as a single dose and in several days, etc. The treatment period will generally have a length proportional to the length of the disease process and the specific 2,4-disubstituted quinoline derivative effectiveness and the patient species being treated.

In the above and below description and appended claims it is to be understood that the forms “a”, “an” and “the” include singular as well as plural references unless the context clearly dictates otherwise. For example, the term “a 2,4-disubstituted quinoline derivative” denotes one or more compounds being the same or different chemical modifications of 2,4-disubstituted quinoline.

Further, it is to be understood that the term “comprising” is intended to mean that the methods and compositions of the invention may include the recited 2,4-disubstitted quinoline derivative but not excluding other substances. The term “consisting essentially of” is used to define methods and compositions that include the recited components but exclude other components that may have an essential significance on the biochemical response resulting from the binding of 2,4-disubstituted quinoline derivative to the receptor. For example, a composition consisting essentially of an 2,4-disubstituted quinoline derivative as the active ingredient and a pharmaceutically acceptable carrier will not include or include only insignificant amounts (amounts that will have an insignificant effect on the activity of the receptor) of other compounds capable of binding to the allosteric site or binding site of the receptor. “Consisting of” shall thus mean excluding more than trace elements of other components. Embodiments defined by each of these transition terms are within the scope of this invention.

Yet further, it is to be understood that all numerical values, e.g. when referring the amounts or ranges of the components constituting the composition of the invention, are approximations which are varied (+) or (−) by up to 20%, at times by up to 10% of from the stated values. It is to be understood, even if not always explicitly stated that all numerical designations are preceded by the term “about”.

The invention will now be described by way of non-limiting examples.

### Some Exemplary Embodiments

### Materials and Methods

### Instruments and Analysis

Microwave-assisted chemistry was performed on an Emrys™ Optimizer with Emrys™ Optimizer software. For the reactions round-bottom vials with a volume of 2-5 mL were used.

¹H-NMR spectra were measured at 200 MHz with a Bruker AC 200 or Bruker DMX 600 spectrometer. ¹³C-NMR spectra were measured at 50 or 150 MHz. Chemical shifts for ¹H and ¹³C are given in ppm (δ) relative to tetramethylsilane (TMS) as internal standard, coupling constants are given in Hz. Melting points were determined with a Büchi capillary melting point apparatus and are uncorrected. Combustion analyses of new target compounds were performed by the analytical department of the Gorlaeus Laboratories, Leiden University (The Netherlands) and are within 0.4% of theoretical values unless otherwise specified.

### Chemical Synthesis

Quinoline-1-oxide (2)

Compound 2 was prepared as described elsewhere [Ochiai, E. Recent Japanese Work on the Chemistry of Pyridine 1-oxide and Related Compounds, J. Org. Chem., 1953, 18, 534-551; Zhong, P. et al. A Simple and Efficient Method for the Preparation of Heterocyclic N-oxide. Synth. Commun. 2004, 34, 247-253]. In brief, to a solution of quinoline (25.83 g, 0.2 mol) in acetic acid (70 mL) was added H₂O₂ (35% in water, 1.5 eq, 29 mL) and the reaction stirred at 70° C. for 21 hours. pH was adjusted to 8 with 2M NaOH and the reaction was extracted with DCM (4×80 mL). The organic layer was dried over MgSO₄ and evaporated. The product was purified by column chromatography, eluent ethyl acetate. The product was crystallized from ethyl acetate. Yield: 21.72 g (72%). ¹H NMR (CDCl₃) δ 7.30 (t, 1H, J=7.66, 6.96 Hz, Ar), 7.61-7.90 (m, 4H, Ar), 8.54 (d, 1H, J=5.84 Hz, Ar), 8.75 (d, 1H, J=8.78 Hz, Ar).

### 4-Nitroquinoline-1-oxide (3)

Compound 3 was prepared by a method described elsewhere [Taylor Jr, E. C. et al. 3-Methyl-4-nitropyridine-1-oxide. Org. Synth. 1963. Coll. Vol. 4. 654-656]. In brief, compound 2 (19.10 g, 0.13 mol) was dissolved in concentrated sulfuric acid and warmed to 65° C. Nitric acid (65%, 1.1 eq, 15 mL) was added slowly, dropwise. The reaction stirred at 65° C. for 2 hours. The reaction was cooled and poured on ice. The product precipitated as yellow solid, which was filtered off, washed with 5% Na₂CO₃ (1×10 mL) water (2×10 mL), ethanol (1×10 mL) and dried. Yield: 21.90 g (88%). ¹H NMR (CDCl₃) δ 7.85-7.94 (m, 2H, Ar), 8.21 (d, 1H, J=6.58 Hz, Ar), 8.53 (d, 1H, J=7.30 Hz, Ar), 8.73-8.86 (m, 1H, Ar). ¹H NMR was identical with ¹H NMR spectrum in literature [Yokoyama, A. et al. Nitration of Quinoline 1-oxide: Mechanism of Regioselectivity. Chem. Pharm. Bull. 1997, 45, 279-283].

### 2-Bromo-4-nitroquinoline (4)

Compound 4 was prepared as described elsewhere [Hamana, M. et al. A new deoxidation reaction of aromatic tertiary amine oxides. Reaction of 4-nitroquinoline 1-oxide with phosphorus bromide. Chem. Abstr. 1957. 51. 6639]. In brief, compound 3 (1.86 g, 9.8 mmol) was dissolved in chloroform and cooled in an ice-bath. POBr₃ (1.25 eq) was added and the reaction stirred in an ice-bath for 2 hours. The reaction was poured on ice, pH was adjusted to 9 with 2M NaOH and extracted with DCM (3×80 mL). The product was purified by column chromatography, eluent DCM. Yield: 1.30 g (52%). ¹HNMR (CDCl₃) δ 7.63-7.94 (m, 2H, Ar), 8.06 (s, 1H, Ar), 8.17 (t, 1H, J—7.16, 6.84 Hz, Ar), 8.38 (d, 1H, J=8.76 Hz, Ar). ¹H NMR was identical with ¹H NMR spectrum in literature [Woźniak, M. et al. Amination of 4-nitroquinoline with Liquid Methylamine/Potassium permanganate. Chem. Heterocyc. Comp. 1998, 34, 837-840].

### 4-Amino-2-bromoquinoline (5)

Compound 5 was prepared by a method described elsewhere [Kornblum, N. et al. The reduction of optically active 2-nitrooctane and α-phenylnitroethane. J. Am. Chem. Soc. 1955. 77. 6266-6269; Den Hertog, H. J.; Buurman, D. J. Rec. Tray. China. des Pays-Bas. 1972, 91, 841-849]. In brief, compound 4 (1.82 g, 7.2 mmol) was dissolved in acetic acid. Iron powder (5 eq) was added and the reaction stirred at 65° C. for 2.5 hours. The iron powder was filtered off, washed with DCM. pH was adjusted to 9 with 2M NaOH. This was filtered again and the residue was washed with ammonia. The aqueous layer was extracted with DCM, dried on MgSO₄ and evaporated. The product was purified by column chromatography, eluent DCM. Yield: 0.69 g (43%). ¹H NMR (CDCl₃) δ 4.81 (bs, 2H, NH₂), 6.76 (s, 1H, Ar), 7.48 (t, 1H, J=7.31, 7.06 Hz, Ar), 7.62-7.73 (m, 2H, Ar), 7.94 (d, 1H, J=8.76 Hz, Ar). ¹³C NMR (CDCl₃) δ 106.60, 117.82, 120.21, 125.37, 129.19, 130.40, 142.59, 148.78, 150.90.

### 2,4-Dichloroquinoline (8)

Compound 8 was prepared as described elsewhere [Osborne, A. G. et al. 2,4-Dihalogenoquinolines. Synthesis, Orientation Effects and ¹H and ¹³C NMR Spectral Studies. J. Chem. Soc. Perkin Trans. I. 1993, 1, 2747-2755]. In brief, malonic acid (8.32 g, 0.08 mol) was dissolved in POCl₃ (60 mL) and cooled in an ice-bath. Aniline (1.25 eq) was added dropwise. The reaction was refluxed for 2.5 hours, then it was cooled to room temperature and poured on ice. pH was adjusted to 9 with 2M NaOH. The precipitate was filtered off and the aqueous layer was extracted with DCM. The product was purified by column chromatography, eluent DCM. Yield: 5.51 g (35%). ¹H NMR (CDCl₃) δ 1.25 (s, 1H, Ar), 7.64 (t, 1H, J=7.84, 6.16 Hz, Ar), 7.79 (t, 1H, J=6.89, 6.71 Hz, Ar), 8.03 (d, 1H, J=8.16 Hz, Ar), 8.21 (d, 1H, J=8.21 Hz, Ar). ¹H NMR spectrum was identical with ¹H NMR spectrum in literature.

### 4-Amino-2-chloroquinoline (9)

### Preparation of compound 9 is described elsewhere [von Büchi, J. et al. Die Tuberkulostatische Wirkung von 2-Oxy-4-amino-chinolin Derivaten. Helv. Chim. Acta. 1949, 32, 1806-1814; Wojahn, H. Untersuchungen über den Zusammenhang von chemischer Konstitution and anästhesierender Wirkung bei 2-Alkoxy-chinolin Deivaten. Arch. Pharm. 1936, 274, 83-106]. However, we prepared compound 9 as described here. In brief, compound 8 (0.35 g, 1.8 mmol) was suspended in ammonia (28-30% in water, 3 mL). The reaction was carried out in the microwave at 160° C. for 2.5 hours. After the reaction was completed, ammonia was evaporated off. The product was purified by column chromatography, eluent 3% MeOH in DCM. Yield: 0.14 g (44%). ¹H NMR (CDCl₃) δ 4.83 (bs, 2H, NH₂), 6.62 (s, 1H, Ar), 7.43-7.51 (m, 1H, Ar), 7.63-7.73 (m, 2H, Ar), 7.89-7.95 (m, 1H, Ar). ¹³C NMR (600 MHz, CDCl₃) δ 103.07, 117.60, 120.08, 125.27, 129.13, 130.46, 148.26, 151.40, 151.41.

### N-(2-bromoquinolin-4-yl)cyclopentanecarboxamide (10)

In brief, compound 5 (0.32 g, 1.40 mmol) was dissolved in pyridine (5 mL) and cyclopenthanecarbonyl chloride (1.3 eq) was added. The reaction stirred at 115° C. for 2 hours. After the reaction was completed, pyridine was evaporated. The product was purified by column chromatography, eluent 5% MeOH in DCM. The product was crystallized from MeOH to give white crystals. Yield: 0.35 g (79%). MS (ESI) m/z: 319.9 [M+H]⁺¹, [M−H]⁺¹. ¹H NMR (CDCl₃) δ 1.64-2.07 (m, 8H, 4CH₂), 2.53-3.00 (m, 1H, CH), 7.54-7.62 (m, 1H, Ar), 7.69-7.79 (m, 2H, Ar), 7.92 (bs, 1H, NH), 7.99-8.08 (m, 1H, Ar), 8.43 (s, 1H, Ar). ¹³C NMR (CDCl₃) δ 25.96, 30.51, 47.2, 114.51, 118.85, 126.76, 129.89, 130.40, 141.56, 143.17, 148.63, 175.07 [Chang, L. C. W. et al. 2,4,6-Trisubstituted pyrimidines as a new class of selective adenosine A₁ receptor antagonists, J. Med. Chem. 2004, 47, 6529-6540].

### General procedure for the preparation of N-(2-chloroquinolin-4-yl)carboxamides (11-15)

In brief, compound 9 was dissolved in pyridine (1 mmol/3 mL) and the appropriate acid chloride (1.3 eq) was added. The reaction was stirred at 60° C. for 90 minutes. After the reaction was completed, pyridine was evaporated. The product was purified by column chromatography [Chang, L. C. W. et al. 2,4,6-Trisubstituted pyrimidines as a new class of selective adenosine A₁ receptor antagonists, J. Med. Chem. 2004, 47, 6529-6540].

### N-(2-chloroquinolin-4-yl)cyclopentanecarboxamide (11)

Scale: 1.6 mmol. Eluent for column chromatography was 5% MeOH in DCM. Yield: 0.34 g (78%). ¹H NMR (CDCl₃) δ 1.58-2.18 (m, 8H, 4CH₂), 2.81-2.98 (m, 1H, CH), 7.53-7.61 (m, 1H, Ar), 7.68-7.76 (m, 2H, Ar), 7.95-8.03 (m, 2H, Ar, NH), 8.42 (s, 1H, Ar). ¹³C NMR (CDCl₃) δ 26.18, 30.72, 47.43, 91.47, 94.32, 111.33, 118.94, 126.86, 130.04, 130.65, 142.41, 148.26, 152.24, 175.28.

### N-(2-chloroquinolin-4-yl)cyclohexanecarboxamide (12)

Scale: 2.2 mmol. Eluent for column chromatography was 2-5% MeOH in DCM. The product was crystallized from MeOH to give white crystals. Yield: 0.60 g (95%). ¹H NMR (CDCl₃) δ 1.28-2.04 (m, 10H, 5CH₂), 2.38-2.53 (m, 1H, CH), 7.56-7.63 (m, 1H, Ar), 7.71-7.79 (m, 2H, Ar), 7.96-7.63 (m, 2H, Ar, NH), 8.44 (s, 1H, Ar). ¹³C NMR (CDCl₃) δ 25.51, 29.66, 46.83, 111.21, 118.52, 118.73, 126.67, 129.89, 130.46, 142.17, 148.05, 152.09, 174.71.

### N-(2-chloro-quinolin-4-yl)benzamide (13)

Scale: 3.91 mmol. Eluent for column chromatography was DCM. Yield: 0.47 g (42%). ¹H NMR (CDCl₃) δ 7.52-7.65 (m, 4H, Ar), 7.72-7.84 (m, 2H, Ar), 7.94-8.06 (m, 3H, Ar), 8.51 (s, 1H, Ar), 8.64 (bs, 1H, NH). ¹³C NMR (CDCl₃) δ 111.40, 118.83, 126.50, 126.89, 128.83, 129.38, 130.26, 132.56, 133.47, 141.93, 147.72, 151.48, 165.67.

### N-(2-chloroquinolin-4-yl)-2-furamide (14)

Scale: 3.36 mmol. Eluent for column chromatography was DCM. Yield: 0.52 g (57%). ¹H NMR (CDCl₃) δ 6.65-6.68 (m, 1H, Ar), 7.39 (d, 1H, J=3.65 Hz, Ar), 7.60-7.90 (m, 4H, Ar), 8.05 (d, 1H, J=12.00 Hz, Ar), 8.52 (s, 1H, Ar), 8.93 (bs, 1H, NH). ¹³C NMR (CDCl₃) δ 110.49, 112.98, 116.77, 118.10, 118.59, 126.60, 129.42, 130.33, 141.36, 144.94, 146.58, 147.67, 151.49, 155.65.

### N-(2-chloroquinolin-4-yl)cyclobutanecarboxamide (15)

Scale: 3.92 mmol. Eluent for column chromatography was 1% MeOH in DCM. Yield: 0.89 g (87%). ¹H NMR (CDCl₃+1 drop of MeOD) S 1.72-2.58 (m, 6H, 3CH₂), 3.28-3.45 (m, 1H, CH), 7.52-7.60 (m, 1H, Ar), 7.69-7.83 (m, 3H, Ar, NH), 7.98-8.03 (m, 1H, Ar), 8.43 (s, 1H, Ar). ¹³C NMR (CDCl₃+1 drop of MeOD) δ 17.85, 25.04, 40.74, 111.13, 118.38, 118.89, 126.35, 129.29, 130.23, 142.09, 147.73, 151.58, 173.81.

### General procedure for the preparation of 2,4-substituted quinolines (16-34)

Method A: Compounds 10-15 were dissolved/suspended in absolute ethanol (1.5 mmol/2.5 mL) and the appropriate amines (3 eq) were added. The mixture was heated in the microwave at 140° C. for 80 min. After the reaction was completed, ethanol was evaporated and the residue was dissolved in DCM (100 mL) and washed with 1 M NaOH (3×100 mL). The organic layer was dried on MgSO₄. The products were purified by column chromatography and recrystallized [Göblyös, A. et al. Structure-activity relationships of new 1H-imidazo[4,5-c]quinolin-4-amine derivatives as allosteric enhancers of the A₃ adenosine receptor, J. Med. Chem. 2006, 49, 3354-3361].

Method B: Compounds 10-15 and the appropriate amines (10 eq) were heated in the microwave without any solvent at 180° C. for 90 min. After the reaction was completed, the reaction mixture was dissolved in DCM (100 mL) and washed with water (2×50 mL), brine (1×50 mL). The organic layer was dried on MgSO₄. The products were purified by column chromatography and recrystallized.

### N-(2-anilinoquinolin-4-yl)cyclopentanecarboxamide (16)

Method A. Scale: 0.36 mmol of compound 10. Eluent for column chromatography was 3-10% MeOH in DCM. The product was recrystallized from methanol to give yellow crystals. Yield: 0.039 g (33%). MS (ESI) m/z: 331.2 [M+H]⁺¹. ¹H NMR (CDCl₃) δ 1.72-2.04 (m, 8H, 4CH₂), 2.80-2.98 (m, 1H, CH), 6.93 (br s, 1H, NH), 7.07 (t, 1H, J=5.74, 8.06 Hz, Ar), 7.25-7.41 (m, 3H, Ar), 7.57-7.67 (m, 5H, m; Ar), 7.78-7.83 (m, 1H, Ar), 8.01 (s, 1H, NH). ¹³C NMR (CDCl₃) δ 25.63, 30.18, 46.89, 100.90, 116.18, 118.36, 119.73, 122.50, 127.25, 129.46, 139.77, 140.96, 147.54, 154.51, 174.87. Anal. calcd for C₂₁H₂₁N₃O.0.3H₂O C, 74.89; H, 6.46; N, 12.48. Found C, 74.89; H, 6.81; N, 12.18.

### N-{2-[(3,4-dichlorophenyl)amino]quinolin-4-yl}cyclopentanecarboxamide (17)

Method B. Scale: 0.34 mmol of compound 10. Eluent for column chromatography was 2% MeOH in DCM. The product was recrystallized from methanol to give white crystals. Yield: 0.024 g (19%). MS (ESI) m/z: 400.3 [M+H]⁺¹. ¹H NMR (CDCl₃) δ 1.61-2.01 (m, 8H, 4CH₂), 2.80-2.97 (m, 1H, CH), 6.79 (bs, 1H, NH), 7.32-7.51 (m, 3H, Ar), 7.57-7.67 (m, 2H, Ar), 7.81-7.94 (m, 3H, Ar, NH), 8.08 (s, 1H, Ar). ¹³C NMR δ (CDCl₃+1 drop of MeOD) δ 25.84, 30.45, 46.40, 102.78, 117.03, 118.30, 119.49, 120.40, 122.97, 124.13, 127.22, 129.68, 130.04, 132.10, 140.41, 141.32, 147.35, 154.21, 176.62. Anal. calcd for C₂₁H₁₉N₃OCl₂.0.55CH₂Cl₂ C, 59.38; H, 4.53; N, 9.40. Found C, 59.00; H, 4.43; N, 9.72.

### N-[2-(benzylamino)quinolin-4-yl]cyclopentanecarboxamide (18)

Method A. Scale: 0.35 mmol of compound 10 and 3 eq of benzylamine.HCl. Eluent for column chromatography was 3-5% MeOH in DCM. The product was recrystallized from methanol to give yellow crystals. Yield: 0.040 g (33%). MS (ESI) m/z: 345.4 [M+H]⁺¹. ¹H NMR (CDCl₃) δ 1.65-2.09 (m, 8H, 4CH₂), 2.79-2.92 (m, 1H, CH), 4.73 (d, 2H, J=8.0 Hz, CH₂), 5.06 (bs, 1H, NH), 7.20-7.41 (m, 7H, Ar), 7.51-7.59 (m, 2H, Ar), 7.70 (s, 1H, Ar), 7.74 (s, 1H, NH). ¹³C NMR (CDCl₃) δ25.66, 30.24, 45.40, 46.92, 100.11, 115.82, 118.27, 121.52, 126.92, 127.10, 127.49, 128.28, 129.25, 139.11, 140.44, 148.42, 157.18, 174.98. Anal. calcd for C₂₂H₂₃N₃O.0.4H₂O C, 74.93; H, 6.80; N, 11.92. Found C, 74.86; H, 6.76; N, 12.16.

### N-{2-[(4-methylphenyl)amino]quinolin-4-yl}cyclopentanecarboxamide (19)

Method A. Scale: 0.36 mmol of compound 10. Eluent for column chromatography was 0-2% MeOH in DCM. The product was recrystallized from methanol to give light brown crystals. Yield: 0.050 g (40%). MS (ESI) m/z: 345.4 [M+H]⁺¹. ¹H NMR (CDCl₃) δ 1.56-2.05 (m, 8H, 4CH₂), 2.31 (s, 3H, CH₃), 2.72-2.88 (m, 1H, CH), 6.87 (bs, 1H, NH), 7.10-7.28 (m, 3H, Ar), 7.43-7.57 (m, 4H, Ar), 7.73-7.89 (m, 3H, Ar, NH). ¹³C NMR (CDCl₃) δ 20.26, 25.42, 29.97, 46.61, 100.53, 116.03, 118.21, 120.03, 122.00, 127.16, 129.10, 131.98, 137.01, 140.56, 147.81, 154.78, 174.77. Anal. calcd. for C₂₂H₂₃N₃O.0.5H₂O C, 74.55; H, 6.82; N, 11.86. Found C, 74.38; H, 6.81; N, 11.94.

### N-[2-(2,3-dihydro-1H-inden-5-ylamino)quinolin-4-yl]cyclopentanecarboxamide (20)

Method A. Scale: 0.35 mmol of compound 10. Fluent for column chromatography was 5% MeOH in DCM. The product was recrystallized from methanol to give brown crystals. Yield: 0.054 g (45%). MS (ESI) m/z: 371.5 [M+H]⁺¹. ¹H NMR (CDCl₃) δ 1.59-2.13 (m, 8H, 4CH₂), 2.74-2.90 (m, 6H, 3CH₂), 6.93 (bs, 1H, NH), 7.14-7.31 (m, 4H, Ar), 7.42-7.58 (m, 3H, Ar), 7.75 (d, 1H, J=8.58 Hz, Ar), 7.88 (s, 1H, NH). ¹³C NMR (CDCl₃) δ 25.60, 25.90, 30.45, 32.27, 33.03, 47.07, 101.02, 116.42, 117.00, 118.64, 118.85, 122.43, 124.58, 127.58, 129.58, 138.05, 139.05, 140.93, 145.23, 148.29, 155.42, 175.22. Anal. calcd. for C₂₄H₂₅N₃O.1.5H₂O C, 72.34; H, 7.08; N, 10.54. Found C, 72.47; H, 6.84; N, 10.32.

### N-{2-[(4-methoxyphenyl)amino]quinolin-4-yl}cyclopentanecarboxamide (21)

Method A. Scale: 0.84 mmol of compound 11. Fluent for column chromatography was 3% MeOH in DCM. The product was recrystallized from methanol to give brown crystals. Yield: 0.12 g (39%). MS (ESI) m/z: 361.4 [M+H]⁺¹. ¹H NMR (CDCl₃) δ 1.60-2.06 (m, 8H, 4CH₂), 2.77-2.95 (m, 1H, CH), 3.82 (s, 3H, CH₃), 6.69 (bs, 1H, NH), 6.88-6.97 (m, 2H, Ar), 7.23-7.31 (m, 1H, Ar), 7.44-7.60 (m, 4H, Ar), 7.73-7.90 (m, 3H, Ar, NH). ¹³C NMR (CDCl₃) δ 25.93, 30.48, 47.25, 55.50, 100.32, 114.48, 116.30, 118.42, 122.43, 122.91, 127.70, 129.68, 133.01, 140.96, 148.45, 155.79, 155.97, 175.10. Anal. calcd. for C₂₂H₂₃N₃O₂.0.5H₂O C, 71.53; H, 6.53; N, 11.34. Found C, 71.55; H, 6.44; N, 11.36.

### N-{2-[(4-chlorophenyl)amino]quinolin-4-yl}cyclopentanecarboxamide (22)

Method A. Scale: 0.96 mmol of compound 11. Eluent for column chromatography was 2-5% MeOH in DCM. The product was recrystallized from methanol to give yellow crystals. Yield: 0.31 g (87%). MS (ESI) m/z: 365.9 [M+H]⁺¹. ¹H NMR (CDCl₃) δ 1.62-216 (m, 8H, 4CH₂), 2.80-2.97 (m, 1H, CH), 6.90 (bs, 1H, NH), 7.27-7.38 (m, 4H, Ar), 7.56-7.59 (m, 4H, Ar), 7.75-7.88 (m, 1H, Ar), 7.98 (s, 1H, NH). ¹³C NMR (CDCl₃) δ 25.96, 30.51, 47.34, 100.96, 116.30, 118.30, 120.85, 123.13, 127.31, 128.01, 129.01, 129.86, 138.80, 141.11, 147.93, 154.42, 175.25. Anal. calcd. for C₂₁H₂₀N₃OCl.0.3CH₂Cl₂ C, 66.29; H, 5.31; N, 10.74. Found C, 66.10; H, 5.28; N, 11.10.

### N-[2-(cyclopentylamino)quinolin-4-yl]cyclopentanecarboxamide (23)

Method A. Scale: 0.93 mmol of compound 11. Eluent for column chromatography was 5% MeOH in DCM. The product was recrystallized from methanol to give off-white crystals. Yield: 0.039 g (14%). MS (ESI) m/z: 323.4 [M+H]⁺¹. ¹H NMR (CDCl₃) δ 1.40-2.16 (m, 16H, 8CH₂), 2.77-2.95 (m, 1H, CH), 4.15-4.32 (m, 1H, CH), 4.92 (bs, 1H, NH), 7.20 (t, 1H, J=9.76 Hz, Ar), 7.48-7.84 (m, 6H, Ar, NH). ¹³C NMR (CDCl₃) δ 23.78, 25.96, 30.51, 33.64, 47.28, 53.20, 99.44, 115.54, 118.39, 121.49, 126.98, 129.58, 140.74, 148.72, 157.70, 175.28. Anal. calcd. for C₂₀H₂₅N₃O.0.7CH₂Cl₂ C, 67.13; H, 6.95; N, 10.97. Found C, 66.87; H, 7.23; N, 11.34.

### N-[2-(1H-indazol-6-ylamino)quinolin-4-yl]cyclopentanecarboxamide (24)

Method A. Scale: 0.87 mmol of compound 11. Eluent for column chromatography was 5-10% MeOH in DCM. The product was recrystallized from methanol to give grey crystals. Yield: 0.15 g (47%). MS (ESI) m/z: 371.4 [M+H]⁺¹. ¹H NMR 300 MHz (CDCl₃+1 drop of MeOD) δ 1.62-2.03 (m, 8H, 4CH₂), 2.84-2.92 (m, 1H, CH), 7.26-7.32 (m, 2H, Ar), 7.43-7.50 (m, 2H, Ar), 7.58 (t, 1H, J=5.33, 4.75 Hz, Ar), 7.67 (d, 1H, J=5.33 Hz, Ar), 7.77 (d, 1H, J=5.40 Hz, Ar), 7.85 (s, 1H, Ar), 8.02 (s, 1H, Ar), 8.08 (s, 1H, Ar). ¹³C NMR (CDCl₃+1 drop of MeOD) 25.84, 30.39, 46.46, 101.62, 110.57, 112.03, 116.85, 119.55, 122.37, 123.40, 126.52, 129.74, 133.25, 133.65, 137.41, 141.47, 147.87, 156.00, 176.31. Anal. calcd. for C₂₂H₂₁N₅O C, 71.13; H, 5.70; N, 18.86. Found C, 71.16; H, 5.75; N, 18.78.

### N-(2-anilinoquinolin-4-yl)cyclohexanecarboxamide (25)

Method A. Scale: 1.04 mmol of compound 12. Eluent for column chromatography was 2-4% MeOH in DCM. The product was recrystallized from methanol to give white crystals. Yield: 0.26 g (73%). ¹H NMR (CDCl₃) δ 1.22-2.12 (m, 10H, 5CH₂), 2.31-2.49 (m, 1H, CH), 6.88 (bs, 1H, NH), 7.04 (t, 1H, J=8.38, 7.62 Hz, Ar), 7.26-7.38 (m, 3H, Ar), 7.54-7.67 (m, 4H, Ar), 7.79 (d, 1H, J=8.22 Hz, Ar), 7.87 (s, 1H, NH), 7.96 (s, 1H, Ar). ¹³C NMR (CDCl₃) δ 25.57, 25.84, 29.33, 44.16, 46.25, 98.56, 115.33, 120.40, 121.06, 122.28, 123.52, 125.19, 129.52, 131.31, 137.68, 141.23, 144.66, 153.91, 175.92. Anal. calcd. for C₂₂H₂₃N₃O.0.3H₂O C, 75.32; H, 6.78; N, 11.98. Found C, 75.14; H, 6.97; N, 11.88.

### N-{2-[(3,4-dichlorophenyl)amino]quinolin-4-yl}cyclohexanecarboxamide (26)

Method B. Scale: 0.17 mmol of compound 12. Eluent for column chromatography was 1% MeOH in DCM. The product was recrystallized from ethyl acetate to give white crystals. Yield: 0.16 g (43%). ¹H NMR (CDCl₃) δ 1.23-2.17 (m, 10H, 5CH₂), 2.35-2.50 (m, 1H, CH), 6.77 (bs, 1H, NH), 7.32-7.41 (m, 2H, Ar), 7.49 (dd, 1H, J=6.22, 2.56 Hz, Ar), 7.58-7.67 (m, 2H, Ar), 7.84-7.88 (m, 2H, Ar, NH), 7.95 (s, 1H, Ar), 8.09 (d, 1H, J=2.56 Hz, Ar). ¹³C NMR (DMSO-d₆) δ 25.23, 25.48, 29.27, 38.30, 44.46, 103.69, 117.58, 118.24, 119.03, 121.55, 122.00, 122.55, 127.07, 129.67, 130.28, 130.80, 141.89, 141.98, 147.50, 154.26, 175.87. Anal, calcd. for C₂₂H₂₁O₂N₃O.0.5H₂O C, 62.42; H, 5.24; N, 9.93. Found C, 62.67; H, 5.20; N, 9.94.

### N-{2-[(4-methylphenyl)amino]quinolin-4-yl}cyclopentanecarboxamide (27)

Method A. Scale: 1.00 mmol of compound 12. Eluent DCM:ethylacetate:MeOH=70:20:10. The product was recrystallized from MeOH:petroleum ether=1:30. Yield: 0.14 g, 39%. ¹H NMR (CDCl₃) δ 1.30-2.09 (m, 10H, 5CH₂), 2.34-2.46 (m, 1H, CH), 2.34 (s, 3H, CH₃), 6.71 (bs, 1H, NH), 7.15-7.35 (m, 4H, Ar), 7.47-7.62 (m, 3H, Ar), 7.79 (d, 1H, J=7.30 Hz, Ar), 7.99 (s, 1H, Ar). ¹³C NMR (DMSO-d₆) δ 20.58, 25.34, 29.46, 46.60, 100.64, 116.25, 118.20, 120.29, 122.32, 127.69, 129.42, 132.27, 137.27, 140.64, 148.25, 155.07, 174.60. Anal. calcd. for C₂₃H₂₅N₃O.0.5H₂O C, 74.97; H, 7.11; N, 11.40. Found C, 74.97; H, 6.96; N, 11.39.

### N-[2-(2,3-dihydro-1H-inden-5-ylamino)quinolin-4-yl]cyclohexanecarboxamide (28)

Method A. Scale: 1.20 mmol of compound 12. Eluent 10-17% ethylacetate in DCM. The product was recrystallized from MeOH:petroleum ether=1:30 to give white crystals. Yield: 0.08 g, 16%. ¹H NMR (CDCl₃) δ 1.35-2.40 (m, 10H, 5CH₂), 2.86-2.98 (m, 1H, CH), 6.72 (s, 1H, NH), 7.19-7.34 (m, 4H, Ar), 7.48-7.62 (m, 2H, Ar), 7.79 (d, 1H, J=8.00 Hz, Ar), 7.97 (s, 1H, Ar). ¹³C NMR (DMSO-d₆) δ 14.16, 21.02, 25.58, 25.65, 29.70, 32.32, 33.08, 46.83, 60.37, 100.86, 116.43, 116.99, 118.42, 118.86, 122.48, 124.63, 127.83, 129.61, 138.11, 139.10, 140.81, 145.28, 148.45, 155.47, 174.78. Anal. calcd. for C₂₅H₂₇N₃O.2.4H₂O C, 71.17; H, 7.98; N, 9.09. Found C, 71.17; H, 7.63; N, 8.69.

### N-(2-anilinoquinolin-4-yl)benzamide (29)

Method A. Scale: 0.71 mmol of compound 13. Eluent for column chromatography was 1-2% MeOH in DCM. The product was recrystallized from methanol to give white crystals. Yield: 0.12 g (49%). ¹H NMR (CDCl₃) δ 6.86 (bs, 1H, NH), 7.07 (t, 1H, J=8.03, 6.57 Hz), 7.29-7.42 (m, 3H, Ar), 7.50-7.69 (m, 8H, Ar), 7.85 (d, 1H, J=8.40 Hz, Ar), 7.93-7.98 (m, 1H, Ar), 8.11 (s, 1H, Ar), 8.50 (bs, 1H, NH). ¹³C NMR (DMSO-d₆) δ 107.03, 118.46, 118.88, 120.97, 122.19, 122.79, 126.79, 128.13, 128.55, 129.61, 132.04, 134.43, 141.65, 141.89, 148.02, 154.66, 166.663. Anal. calcd. for C₂₂H₁₇N₃O C, 77.86; H, 5.05; N, 12.38. Found C, 77.56; H, 5.20; N, 12.46.

### N-{2-[(3,4-dichloro-phenyl)amino]quinolin-4-yl}benzamide (30)

Method B. Scale: 0.94 mmol of compound 13. Eluent for column chromatography was 0.25% MeOH in DCM. The product was recrystallized from methanol to give white crystals. Yield: 0.16 g (42%). ¹H NMR (CDCl₃) δ 6.85 (bs, 1H, NH), 7.39 (t, 2H, J=8.40, 8.04 Hz, Ar), 7.49-7.71 (m, 6H, Ar), 7.87-7.99 (m, 3H, Ar), 8.09-8.13 (m, 2H, Ar), 8.54 (bs, 1H, NH). ¹³C NMR (DMSO-d₆) δ 106.45, 118.34, 118.85, 119.12, 121.76, 122.79, 126.95, 128.16, 128.52, 129.86, 130.37, 130.86, 132.10, 134.40, 141.77, 142.32, 147.59, 154.08, 166.75. Anal. calcd. for C₂₂H₁₅Cl₂N₃O C, 64.72; H, 3.70; N, 10.29. Found C, 64.42; H, 3.85; N, 10.34.

### N-(2-anilinoquinolin-4-yl)-2-furamide (31)

Method A. Scale: 0.58 mmol of compound 14. Eluent for column chromatography was 1-2% MeOH in DCM. The product was recrystallized from methanol to give white crystals. Yield: 0.058 g (32%). ¹H NMR (CDCl₃) δ 6.52-6.54 (m, 1H, Ar), 7.02 (t, 1H, J=7.30 Hz, Ar), 7.24-7.35 (m, 6H, Ar), 7.51-7.62 (m, 5H, Ar, NH), 8.09 (s, 1H Ar), 8.74 (bs, 1H, NH). ¹³C NMR (CDCl₃) δ 101.06, 112.74, 116.19, 118.22, 119.47, 122.19, 122.62, 127.74, 128.77, 129.47, 139.93, 140.17, 144.57, 147.02, 148.05, 154.63, 156.12. Anal. calcd. for C₂₀H₁₅N₃O₂ C, 72.93; H, 4.59; N, 12.75. Found C, 72.35; H, 4.61; N, 12.59.

### N-{2-[(3,4-dichlorophenyl)amino]quinolin-4-yl}-2-furamide (32)

Method B. Scale: 0.77 mmol of compound 14. Eluent for column chromatography was 0-1% MeOH in DCM. The product was recrystallized from methanol to give white crystals. Yield: 0.18 g (59%). ¹H NMR (CDCl₃) δ 6.58-6.61 (m, 1H, Ar), 7.26-7.50 (m, 4H, Ar), 7.59-7.83 (m, 4H, Ar), 8.08 (s, 1H, Ar), 8.15 (s, 1H, Ar). ¹³C NMR (DMSO-d₆) δ 106.42, 112.36, 115.88, 118.37, 118.64, 119.19, 121.82, 122.61, 122.85, 126.95, 129.92, 130.34, 130.86, 141.50, 141.71, 146.44, 146.95, 147.56, 153.99, 156.96. Anal. calcd. for C₂₀H₁₃Cl₂N₃O₂ C, 60.23; H, 3.29; N, 10.55. Found C, 60.10; H, 3.48; N, 10.59.

### N-(2-anilinoquinolin-4-yl)cyclobutanecarboxamide (33)

Method B. Scale: 0.77 mmol of compound 15. Eluent for column chromatography was 1-2% MeOH in DCM. The product was recrystallized from ethyl acetate to give white crystals. Yield: 0.17 g (71%). ¹H NMR (CDCl₃) δ 1.93-2.56 (m, 6H, 3CH₂), 3.24-3.41 (m, 1H, CH), 6.84 (bs, 1H, NH), 7.05 (t, 1H, J=7.30 Hz, Ar), 7.26-7.40 (m, 3H, Ar), 7.53-7.82 (m, 6H, Ar, NH), 7.99 (s, 1H, NH). ¹³C NMR (CDCl₃) δ 17.75, 25.11, 40.64, 101.94, 116.52, 118.74, 119.40, 122.10, 122.38, 127.41, 128.74, 129.35, 140.32, 140.50, 147.90, 154.63, 174.28. Anal. calcd. for C₂₀H₁₉N₃O.0.5H₂O C, 73.60; H, 6.18; N, 12.87. Found C, 73.94; H, 6.15; N, 12.95.

### N-{2-[(3,4-dichlorophenyl)amino]quinolin-4-yl}cyclobutanecarboxamide (34)

Method B. Scale: 0.77 mmol of compound 15. Eluent for column chromatography was 0-0.5% MeOH in DCM. The product was recrystallized from methanol to give white crystals. Yield: 0.13 g (44%). ¹H NMR (CDCl₃) δ 1.95-2.53 (m, 6H, 3CH₂), 3.23-3.42 (m, 1H, CH), 6.79 (bs, 1H, NH), 7.31-7.39 (m, 2H, Ar), 7.47-7.70 (m, 5H, Ar, NH), 7.86 (d, 1H, J=8.40 Hz, Ar), 7.95 (s, 1H, Ar), 8.09 (s, 1H, Ar). ¹³C NMR (DMSO-d₆) 17.90, 24.78, 103.57, 117.49, 118.25, 119.06, 121.58, 121.94, 122.58, 127.10, 129.70, 130.28, 130.80, 141.89, 141.95, 147.47, 154.29, 174.45. Anal. calcd. for C₂₀H₁₇Cl₂N₃O C, 62.19; H, 4.44; N, 10.88. Found C, 61.85; H, 4.65; N, 10.82.

Table 1 summarizes the chemical structures and physico-chemical characteristics of the 2,4-disubstituted quinoline derivatives prepared as described above.

TABLE 1
Chemical structures and physico-chemical characteristics

| | | Molecular | | Mp | | Recrystallization |
|---|---|---|---|---|---|---|
| Nr | Compound | Formula | MW | (° C.) | MS | solvent |
| 16 | 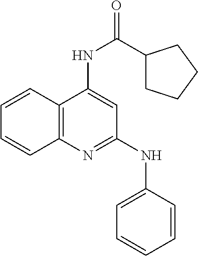 | C₂₁H₂₁N₃O | 331.42 | 173-174 | 332.2 | MeOH |
| 17 | 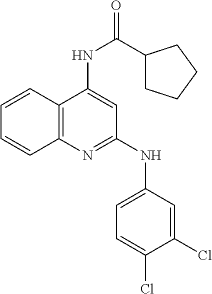 | C₂₁H₁₉N₃OCl₂ | 400.31 | 216-217 | 400.2 | MeOH |
| 18 | 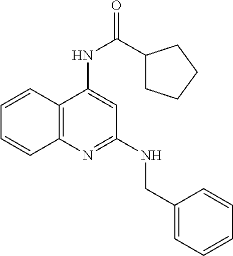 | C₂₂H₂₃N₃O | 345.45 | 154-155 | 345.9 | MeOH |
| 19 | 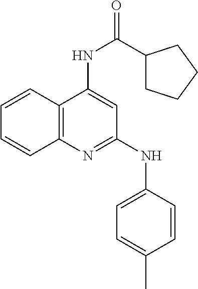 | C₂₂H₂₃N₃O | 345.45 | 188-190 | 345.5 | MeOH |
| 20 | 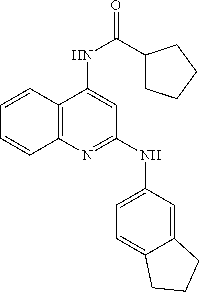 | C₂₄H₂₅N₃O | 371.49 | 115-117 | 372.2 | MeOH |
| 21 | 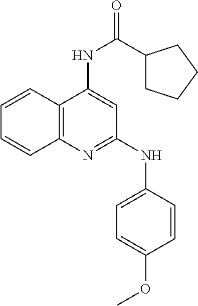 | C₂₂H₂₃N₃O₂ | 361.45 | 191-192 | 362.1 | MeOH |
| 22 | 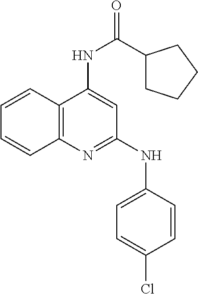 | C₂₁H₂₀N₃OCl | 365.87 | 198-199 | 366.1 | MeOH |
| 23 | 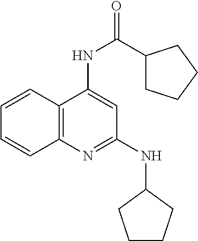 | C₂₀H₂₅N₃O | 323.44 | 183-184 | 324.0 | MeOH |
| 24 | 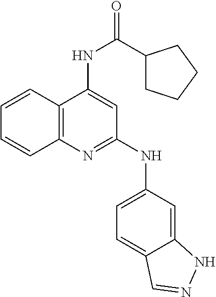 | C₂₂H₂₁N₅O | 371.45 | 222-224 | 371.9 | MeOH |
| 25 | 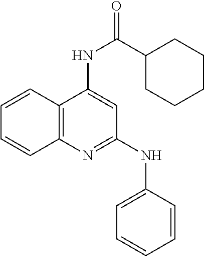 | C₂₂H₂₃N₃O | 345.45 | 191-192 | — | MeOH |
| 26 | 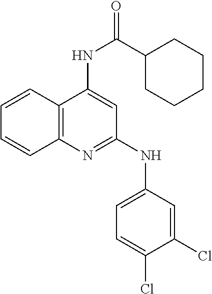 | C₂₂H₂₁Cl₂N₃O | 314.34 | 226-227 | — | EA |
| 27 | 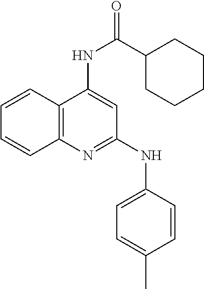 | C₂₃H₂₅N₃O | 359.20 | 189-190 | 360.27 | MeOH/PE |
| 28 | 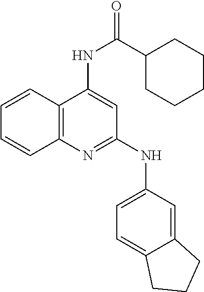 | C₂₅H₂₇N₃O | 385.22 | 175-176 | 386.27 | MeOH/PE |
| 29 | 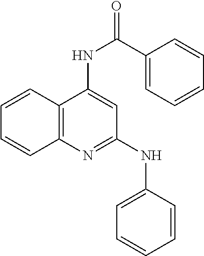 | C₂₂H₁₇N₃O | 339.40 | 223-224 | — | MeOH |
| 30 | 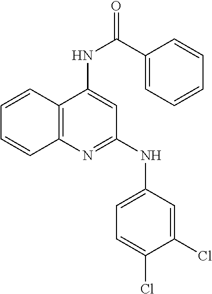 | C₂₂H₁₅Cl₂N₃O | 408.29 | 224-225 | — | MeOH |
| 31 | 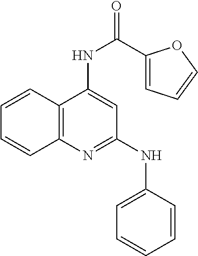 | C₂₀H₁₅N₃O₂ | 329.36 | 215-216 | — | MeOH |
| 32 | 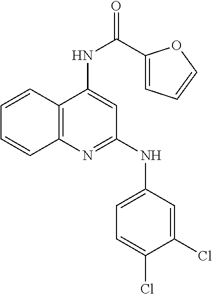 | C₂₀H₁₃Cl₂N₃O₂ | 398.25 | 217-218 | — | MeOH |
| 33 | 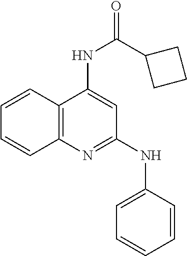 | C₂₀H₁₉N₃O | 317.39 | 154-155 | — | EA |
| 34 | 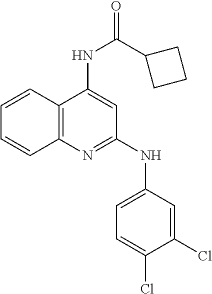 | C₂₀H₁₇Cl₂N₃O | 386.28 | 193-194 | — | MeOH |

MW: molecular weight (D);
Mp: melting point (° C.);
MS: mass spectrometry data (M + H)

### Biological Experiments

### Materials:

[¹²⁵I]N⁶-(4-amino-3-iodobenzyl)adenosine-5′-N-methyluronamide (I-AB-MECA; 2000 Ci/mmol), was from Amersham Pharmacia Biotech (Buckinghamshire, UK).

### Cell Culture and Membrane Preparation:

CHO (Chinese hamster ovary) cells expressing the recombinant human A₃ receptors were cultured in DMEM and F12 (1:1) supplemented with 10% fetal bovine serum, 100 units/ml penicillin, 100 μg/ml streptomycin and 2 μmol/ml glutamine. Cells were harvested by trypsinization. After homogenization and suspension, cells were centrifuged at 500 g for 10 min, and the pellet was re-suspended in 50 mM Tris.HCl buffer (pH 7.4) containing 10 mM MgCl₂. The suspension was homogenized with an electric homogenizer for 10 sec, and was then re-centrifuged at 20,000 g for 20 mM at 4° C. The resultant pellets were resuspended in buffer in the presence of 3 Units/mL adenosine deaminase, and the suspension was stored at −80° C. until the binding experiments. The protein concentration was measured using the Bradford assay.

### Binding Assay to the Human A₃ AR:

Each tube in the competitive binding assay contained 100 μl membrane suspension (20 μg protein), 50 μl [¹²⁵I]I-AB-MECA (0.5 nM), and 50 μl of increasing concentrations of the test modulators in Tris.HCl buffer (50 mM, pH 8.0) containing 10 mM MgCl₂, 1 mM EDTA. Nonspecific binding was determined using 10 μM of 5′-N-ethylcarboxamidoadenosine in the buffer. The mixtures were incubated at 25° C. for 60 min. Binding reactions were terminated by filtration through Whatman GF/B filters under reduced pressure using a MT-24 cell harvester (Brandell, Gaithersburgh, Md., USA). Filters were washed three times with 9 mL ice-cold buffer. Radioactivity was determined in a Beckman 5500B γ-counter.

Dissociation Kinetics of [¹²⁵I]I-AB-MECA from Human A₃ARs:

The dissociation of [¹²⁵I]I-AB-MECA was measured as follows. Membranes (20 μg) were pre-incubated at 25° C. with 0.5 nM [¹²⁵I]I-AB-MECA, in a total volume of 100 μl of Tris-HCl buffer (50 mM, pH 8.0) containing 10 mM MgCl₂, and 1 mM EDTA for 60 min. The dissociation was then initiated by the addition of 3 μM Cl-IB-MECA with or without allosteric modulators. The time course of dissociation of total binding was measured by rapid filtration at appropriate time intervals. Nonspecific binding was measured after 60-min incubation in the presence of 3 μM Cl-IB-MECA. Further assay was as described above.

### Statistical Analysis

Binding parameters were calculated using Prism 5.0 software (GraphPAD, San Diego, Calif., USA). IC₅₀ values obtained from competition curves were converted to Kᵢ values using the Cheng-Prusoff equation. Data were expressed as mean±standard error.

TABLE 2
Potency of N-[(2-amino)quinolin-4-yl]carboxamide
derivatives in binding assays at human A₃ARs and
allosteric effects at the human A₃AR

| | | | % displace- | hA₃ARAg |
|---|---|---|---|---|
| | | | ment at | Dissoci- |
| | | | 10 μM | ation^{b} |
| No. | R₁ | R₂ | (hA₃AR)^{a} | at 10 μM |
| 16 | c-pent | Ph | 61 | 145 |
| | | | (56/66) | (142/147) |
| 17 | c-pent | 3,4-Cl₂—Ph | 50 | 169 |
| | | | (45/55) | (168/170) |
| 18 | c-pent | PhCH₂ | 28 | 107 |
| | | | (23/34) | (105/108) |
| 19 | c-pent | 4-Me—Ph | 63 | 165 |
| | | | (61/64) | (160/170) |
| 20 | c-pent | 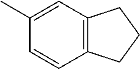 | 62 (60/64) | 147 (143/150) |
| 21 | c-pent | 4-OMe—Ph | 35 | 134 |
| | | | (24/45) | (131/137) |
| 22 | c-pent | 4-Cl—Ph | 28 | 171 |
| | | | (18/38) | (169/173) |
| 23 | c-pent | c-pent | 14 | 114 |
| | | | (9/19) | (113/114) |
| 24 | c-pent | 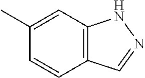 | 90 (88/91) | 102 (101/102) |
| 25 | c-hex | Ph | 26 | 210 |
| | | | (24/28) | (205/214) |
| 26 | c-hex | 3,4-Cl₂—Ph | 17 | 249 |
| | | | (16/17) | (264/234) |
| 27 | c-hex | 4-Me—Ph | 64 | 220 |
| | | | (63/65) | (228/211) |
| 28 | c-hex | 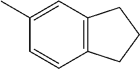 | 42 (37/46) | 217 (213/221) |
| 29 | Ph | Ph | 76 | 100 |
| | | | (76/76) | (98/101) |
| 30 | Ph | 3,4-Cl₂—Ph | 37 | 112 |
| | | | (36/38) | (106/117) |
| 31 | 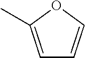 | Ph | 60 (60/60) | 100 (98/101) |
| 32 | 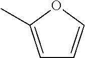 | 3,4-Cl₂—Ph | 13 (7/18) | 96 (93/99) |
| 33 | c-but | Ph | 66 | 119 |
| | | | (64/69) | (116/122) |
| 34 | c-but | 3,4-Cl₂—Ph | 51 | 149 |
| | | | (49/52) | (149/149) |

^{a}All experiments were performed using adherent CHO (A₃), cells stably transfected with cDNA encoding the human ARs. Binding at human A₃ARs in this study was carried out as described in Methods using [¹²⁵I]I-AB-MECA (0.1 nM) as a radioligand. Values from the present study are expressed as mean ± s.e.m., n = 3-5. Percentage inhibition at A₃ receptors is expressed as the mean value from 2-4 separate experiments with similar results performed in duplicate.
^{b}Dissociation by Cl-AB-MECA in the absence of modulator (control) was set at 100%. Increase in binding of control was determined after 1.5 or 2 hours of dissociation by Cl-AB-MECA in presence of 10 μM of the test compounds, respectively. Values are means of two separate assays performed in duplicate (within brackets are the result of each of the two assays).

In Table 2 the effects of the 2,4-disubstituted quinoline derivatives at the orthosteric site of the human adenosine A₃ receptor are listed (column 4), together with their effects on the allosteric site on the human adenosine A₃ receptor (column 5). Many compounds display little if any affinity for the orthosteric binding site, especially when R₁=3,4-Cl₂-phenyl (≦50% displacement). The best separation between orthosteric and allosteric recognition was found with compound 26 with little displacement at the orthosteric site (17%) and a huge allosteric effect (247% vs the control value of 100%). Most compounds significantly retard the dissociation of the radioligand.

1.-47. (canceled)

48. A method comprising administering to a subject an A₃ adenosine receptor allosteric modulator (A₃RM), having the following general formula (I): 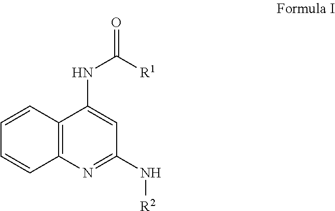
wherein:
R₁ is a group selected from C₄-C₁₂ cycloalkyl, C₄-C₁₂ cycloalkenyl, C₆-C₁₂ aryl, C₄-C₁₂ heteroaryl, alkcycloalkyl, alkaryl, C₁-C₁₀ alkyl, C₂-C₁₀ alkenyl, C₂-C₁₀ alkynyl, C₅-C₁₅ fused cycloalkyl, bicyclic aromatic or heteroaromatic ring, C₁-C₁₀ alkylether, amino, hydrazido, C₁-C₁₀ alkylamino, C₁-C₁₀ alkoxy, C₁-C₁₀ alkoxycarbony, C₁-C₁₀ alkanol, C₁-C₁₀ acyl, C₁-C₁₀ thioalkoxy, pyridylthio, thio, and C₁-C₁₀ alkylthio, acetoamido and sulfonic acid;
R₂ is a group selected from hydrogen or a cyclic moiety selected from the group consisting of aryl, heteroary, alkaryl, alkheteroaryl, cycloalkyl, heterocycloalkyl, cycloal kenyl, heterocycloalkenyl, cycloalkynyl, heterocycloal kynyl, al kcycloal kyl, al kcycloheteroal kyl, al kcycloal kenyl, and alkheterocycloalkenyl, said cyclic moiety optionally substituted by at least one group selected from C₁-C₁₀ alkyl, halo, C₁-C₁₀ alkanol, hydroxyl, C₁-C₁₀ acyl, C₁-C₁₀ alkoxy, C₁-C₁₀ alkoxycarbony, C₁-C₁₀ alkoxylalkyl, C₁-C₁₀ thioalkoxy, C₁-C₁₀ alkylether, amino, hydrazido, C₁-C₁₀ alkylamino, pyridylthio, C₂-C₁₀ alkenyl, C₂-C₁₀ alkynyl, thio, C₁-C₁₀ alkylthio, acetoamido and sulfonic acid; or said substituents can form together with an atom of a cyclic moiety a cycloalkyl, heterocycloalkyl, cycloalkenyl or heterocycloalkenyl fused to said cyclic moiety;
and pharmaceutically acceptable salts thereof,
wherein, said method is for treatment of a condition which is treatable by a compound selected from the group consisting of adenosine, an A₃ adenosine receptor agonist, or an A₃ adenosine receptor antagonist.

49. The method as claimed in claim 48, wherein R¹ is selected from cycloalkyl, aryl, and heteroaryl, and R² is selected from aryl, alkaryl, and cycloalkyl, the aryl or cycloalkyl being optionally substituted by at least one substituent selected from a C₁-C₁₀ alkyl, halo, and C₁-C₁₀ alkylether.

50. The method as claimed in claim 48, wherein R¹ is selected from C₄-C₆ cycloalkyl, phenyl or a five membered heterocyclic aromatic ring having the following formula (II): 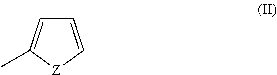
wherein Z is selected from O, S, or NH; and
R² is selected from C₄-C₆ cycloalkyl, phenyl, alkphenyl, or an aromatic ring fused to a five membered cyclic or heteroaromatic ring having the following formulae (IIIa) or (IIIb): 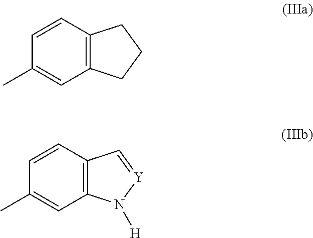
wherein Y is selected from N or CH;
the aryl or cycloalkyl ring in said cycloalkyl, phenyl, alkphenyl or in formulae (Va) or (Vb) being optionally substituted with a substituent selected from C₁-C₁₀ alkyl, halo, or C₁-C₁₀ alkylether.

51. The method as claimed in claim 48, being a 2,4-disubstituted quinoline derivative selected from:
N-(2-anilinoquinolin-4-yl)cyclopentanecarboxamide,
N-{2-[(3,4-dichlorophenyl)amino]quinolin-4-yl}cyclopentanecarboxamide,
N-[2-(benzylamino)quinolin-4-yl]cyclopentanecarboxamide,
N-{2-[(4-methylphenyl)amino]quinolin-4-yl}cyclopentanecarboxamide,
N-[2-(2,3-dihydro-1H-inden-5-ylamino)quinolin-4-yl]cyclopentanecarboxamide,
N-{2-[(4-methoxyphenyl)amino]quinolin-4-yl}cyclopentanecarboxamide,
N-{2-[(4-chlorophenyl)amino]quinolin-4-yl}cyclopentanecarboxamide,
N-[2-(cyclopentylamino)quinolin-4-yl]cyclopentanecarboxamide,
N-[2-(1H-indazol-6-ylamino)quinolin-4-yl]cyclopentanecarboxamide,
N-(2-anilinoquinolin-4-yl)cyclohexanecarboxamide,
N-{2-[(3,4-dichlorophenyl)amino]quinolin-4-yl}cyclohexanecarboxamide,
N-{2-[(4-methylphenyl)amino]quinolin-4-yl}cyclohexanecarboxamide,
N-[2-(2,3-dihydro-1H-inden-5-ylamino)quinolin-4-yl]cyclohexanecarboxamide,
N-(2-anilinoquinolin-4-yl)benzamide,
N-{2-[(3,4-dichloro-phenyl)amino]quinolin-4-yl}benzamide,
N-(2-anilinoquinolin-4-yl)-2-furamide,
N-{2-[(3,4-dichlorophenyl)amino]quinolin-4-yl}-2-furamide,
N-(2-anilinoquinolin-4-yl)cyclobutanecarboxamide, and
N-{2-[(3,4-dichlorophenyl)amino]quinolin-4-yl}cyclobutanecarboxamide.

52. The method as claimed in claim 48, for enhancing the activity of an A₃ adenosine receptor (A₃AR) thereby treating a condition treatable by adenosine or an A₃AR agonist.

53. The method as claimed in claim 52, wherein R¹ is a C₄-C₆ cycloalkyl or a phenyl; and
R² is selected from C₄-C₆ cycloalkyl, phenyl, or an aromatic ring fused to a five membered cycloalkyl having the following formulae (III): 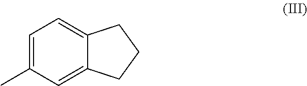
the phenyl moiety in R² being unsubstituted or substituted at least once with a C₁-C₃ alkyl, halogen, or C₁-C₃ alkether.

54. The A₃RM as claimed in claim 53, wherein said R¹ is selected from cyclopentyl, cyclohexyl, cyclo butyl, or phenyl; and
R² is selected from cyclopentyl, phenyl, or an aromatic ring fused to a five membered cycloalkyl having the following formulae (III): 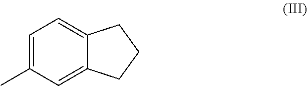
the phenyl moiety in R² being unsubstituted or substituted at least once with a methyl, Cl, or methylether.

55. The A₃RM as claimed in claim 54, being a 2,4-disubstituted quinoline derivative selected from:
N-{2-[(3,4-dichlorophenyl)amino]quinolin-4-yl}cyclopentanecarboxamide N-{2-[(4-methoxyphenyl)amino]quinolin-4-yl}cyclopentanecarboxamide,
N-{2-[(4-chlorophenyl)amino]quinolin-4-yl}cyclopentanecarboxamide,
N-(2-anilinoquinolin-4-yl)cyclohexanecarboxamide,
N-{2-[(3,4-dichlorophenyl)amino]quinolin-4-yl}cyclohexanecarboxamide,
N-{2-[(4-methylphenyl)amino]quinolin-4-yl}cyclohexanecarboxamide,
N-[2-(2,3-dihydro-1H-inden-5-ylamino)quinolin-4-yl]cyclohexanecarboxamide, and
N-{2-[(3,4-dichlorophenyl)amino]quinolin-4-yl}cyclobutanecarboxamide.

56. The method as claimed in claim 54, being selected from
N-{2-[(4-chlorophenyl)amino]quinolin-4-yl}cyclopentanecarboxamide,
N-(2-anilinoquinolin-4-yl)cyclohexanecarboxamide,
N-{2-[(3,4-dichlorophenyl)amino]quinolin-4-yl}cyclohexanecarboxamide, and
N-[2-(2,3-dihydro-1H-inden-5-ylamino)quinolin-4-yl]cyclohexanecarboxamide.

57. The method as claimed in claim 56, being N-{2-[(3,4-dichlorophenyl)amino]quinolin-4-yl}cyclohexanecarboxamide.

58. The method as claimed in claim 52, comprising an occurrence of one or more of the following:
(a) an increase of at least 15% in efficacy of said A₃AR by binding of said A₃AR enhancer to an allosteric site of said A₃AR; and
(b) a decrease in dissociation rate of adenosine or an A₃AR agonist from its binding site.

59. The method of claim 58, wherein said enhancement comprises less than 60% of displacement of adenosine or an A₃AR agonist from its binding site.

60. The method as claimed in claim 52, wherein said condition being treatable by an adenosine or an A₃AR agonist is selected from the group consisting of a malignancy, an immuno-compromised affliction, high intraocular pressure, and a condition associated with high intraocular pressure.

61. The method as claimed in claim 52, comprising administration of an agonist to the orthosteric binding site of said A₃AR in combination with administration of the A₃RM.

62. A method comprising administering to a subject an amount of an A₃ adenosine receptor allosteric modulator (A₃RM), the amount being effective to modulate A₃AR activity, wherein said A₃RM has the following general formula (I): 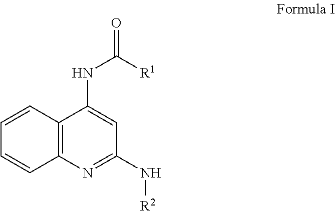
wherein:
R₁ is a group selected from C₄-C₁₂ cycloalkyl, C₄-C₁₂ cycloalkenyl, C₆-C₁₂ aryl, C₄-C₁₂ heteroaryl, alkcycloalkyl, alkaryl, C₁-C₁₀ alkyl, C₂-C₁₀ alkenyl, C₂-C₁₀ alkynyl, C₅-C₁₅ fused cycloalkyl, bicyclic aromatic or heteroaromatic ring, C₁-C₁₀ alkylether, amino, hydrazido, C₁-C₁₀ alkylamino, C₁-C₁₀ alkoxy, C₁-C₁₀ alkoxycarbony, C₁-C₁₀ alkanol, C₁-C₁₀ acyl, C₁-C₁₀ thioalkoxy, pyridylthio, thio, and C₁-C₁₀ alkylthio, acetoamido, and sulfonic acid;
R₂ is a group selected from hydrogen or a cyclic moiety selected from the group consisting of aryl, heteroary, alkaryl, alkheteroaryl, cycloalkyl, heterocycloalkyl, cycloal kenyl, heterocycloalkenyl, cycloalkynyl, heterocycloal kynyl, al kcycloal kyl, al kcycloheteroal kyl, al kcycloal kenyl, and alkheterocycloalkenyl, said cyclic moiety optionally substituted by at least one group selected from C₁-C₁₀ alkyl, halo, C₁-C₁₀ alkanol, hydroxyl, C₁-C₁₀ acyl, C₁-C₁₀ alkoxy, C₁-C₁₀ alkoxycarbony, C₁-C₁₀ alkoxylalkyl, C₁-C₁₀ thioalkoxy, C₁-C₁₀ alkylether, amino, hydrazido, C₁-C₁₀ alkylamino, pyridylthio, C₂-C₁₀ alkenyl, C₂-C₁₀ alkynyl, thio, C₁-C₁₀ alkylthio, acetoamido, and sulfonic acid; or said substituents can form together with an atom of a cyclic moiety a cycloalkyl, heterocycloalkyl, cycloalkenyl, or heterocycloalkenyl fused to said cyclic moiety;
and pharmaceutically acceptable salts thereof;
the method is for affecting an A₃ adenosine receptor (A₃AR) activity in a subject, the effect on the receptor being similar to that of a compound selected from the group consisting of adenosine, an A₃AR agonist and an A₃AR antagonist.

63. A 2,4-disubstituted quinoline derivative selected from the group consisting of:
N-(2-anilinoquinolin-4-yl)cyclopentanecarboxamide,
N-{2-[(3,4-dichlorophenyl)amino]quinolin-4-yl}cyclopentanecarboxamide,
N-[2-(benzylamino)quinolin-4-yl]cyclopentanecarboxamide,
N-{2-[(4-methylphenyl)amino]quinolin-4-yl}cyclopentanecarboxamide,
N-[2-(2,3-dihydro-1H-inden-5-ylamino)quinolin-4-yl]cyclopentanecarboxamide,
N-{2-[(4-methoxyphenyl)amino]quinolin-4-yl}cyclopentanecarboxamide,
N-{2-[(4-chlorophenyl)amino]quinolin-4-yl}cyclopentanecarboxamide,
N-[2-(cyclopentylamino)quinolin-4-yl]cyclopentanecarboxamide,
N-[2-(1H-indazol-6-ylamino)quinolin-4-yl]cyclopentanecarboxamide,
N-(2-anilinoquinolin-4-yl)cyclohexanecarboxamide,
N-{2-[(3,4-dichlorophenyl)amino]quinolin-4-yl}cyclohexanecarboxamide,
N-{2-[(4-methylphenyl)amino]quinolin-4-yl}cyclohexanecarboxamide,
N-[2-(2,3-dihydro-1H-inden-5-ylamino)quinolin-4-yl]cyclohexanecarboxamide,
N-(2-anilinoquinolin-4-yl)benzamide,
N-{2-[(3,4-dichloro-phenyl)amino]quinolin-4-yl}benzamide,
N-(2-anilinoquinolin-4-yl)-2-furamide,
N-{2-[(3,4-dichlorophenyl)amino]quinolin-4-yl}-2-furamide,
N-(2-anilinoquinolin-4-yl)cyclobutanecarboxamide, and
N-{2-[(3,4-dichlorophenyl)amino]quinolin-4-yl}cyclobutanecarboxamide.

64. The 2,4-disubstituted quinoline derivative of claim 63, selected from the group consisting of:
N-{2-[(3,4-dichlorophenyl)amino]quinolin-4-yl}cyclopentanecarboxamide N-{2-[(4-methoxyphenyl)amino]quinolin-4-yl}cyclopentanecarboxamide,
N-{2-[(4-chlorophenyl)amino]quinolin-4-yl}cyclopentanecarboxamide,
N-(2-anilinoquinolin-4-yl)cyclohexanecarboxamide,
N-{2-[(3,4-dichlorophenyl)amino]quinolin-4-yl}cyclohexanecarboxamide,
N-{2-[(4-methylphenyl)amino]quinolin-4-yl}cyclohexanecarboxamide,
N-[2-(2,3-dihydro-1H-inden-5-ylamino)quinolin-4-yl]cyclohexanecarboxamide, and
N-{2-[(3,4-dichlorophenyl)amino]quinolin-4-yl}cyclobutanecarboxamide.

65. The 2,4-disubstituted quinoline derivative of claim 64, selected from the group consisting of:
N-{2-[(4-chlorophenyl)amino]quinolin-4-yl}cyclopentanecarboxamide,
N-(2-anilinoquinolin-4-yl)cyclohexanecarboxamide,
N-{2-[(3,4-dichlorophenyl)amino]quinolin-4-yl}cyclohexanecarboxamide, and
N-[2-(2,3-dihydro-1H-inden-5-ylamino)quinolin-4-yl]cyclohexanecarboxamide.

66. A method of treatment, comprising administration to a subject in need of treatment a 2,4-disubstituted quinoline derivative as claimed in claim 63.

67. A pharmaceutical composition comprising as an active ingredient a 2,4-disubstituted quinoline derivative as claimed in claim 63.
